(19) United States
(12) Patent Application Publication
(10) Pub. No.: US 2026/0276652 A1
(43) Pub. Date: Sep. 17, 2026

(54) METHODS FOR ASSESSING PLATELET FUNCTION

(71) Applicant: UNIVERSITY OF READING, Reading, Berkshire (GB)

(72) Inventors: Jonathan Gibbins, Reading, Berkshire (GB); Christopher Jones, Corsham, Wiltshire (GB); Alex Bye, Reading, Berkshire (GB); Joanne Dunster, Holloway, Derbyshire (GB); Neline Kriek, Slough, Berkshire (GB)

(21) Appl. No.: 19/167,758

(22) PCT Filed: Mar. 22, 2024
(86) PCT No.: PCT/GB2024/050791

§ 371 Date: Sep. 22, 2025

(30) Foreign Application Priority Data
Mar. 23, 2023 (GB) 2304265.8

(51) Int. Cl. G01N 33/68; A61K 38/39

(52) U.S. Cl. CPC G01N 33/6887; A61K 38/39; G01N 2333/70503; G01N 2333/70596; G01N 2800/324; G01N 2800/50; G01N 2800/52

(57) ABSTRACT

The present application relates generally to methods for assessing platelet function, for example in a sample from a subject. These methods can be used to identify subjects based on their platelet reactivity or sensitivity to a platelet activating substance, such as collagen-related peptide (CRP), or a thrombin, ADP, thromboxane A2 or adrenaline receptor agonist. The application also provides methods that involve assessing the level of CD36 in platelets of a subject, the level of GPVI in platelets of the subject, and/or the age of the subject as indicators of the platelet function in that subject. The methods can be used to predict the responsiveness of a subject to anti-platelet therapy and so can form part of a therapeutic method.

### FIELD OF THE INVENTION

The present invention relates generally to methods for assessing platelet function, for example in a sample from a subject. These methods can be used to identify subjects based on their platelet reactivity or sensitivity to a platelet activating substance, such as collagen-related peptide (CRP), or a thrombin, ADP, thromboxane A2 or adrenaline receptor agonist. These methods can also be used to predict the responsiveness of a subject to anti-platelet therapy and so can form part of a therapeutic method.

### BACKGROUND

Platelets, or thrombocytes, are blood cells that help control bleeding. For example, platelets will aggregate at the site of a wound to form a blood clot (platelet aggregation), which can seal blood vessels to prevent or limit bleeding. Thus, platelets help protect from adverse bleeding, such as the effects of injury.

Platelets are involved in the pathology of a number of bleeding conditions and disorders and abnormal platelet levels or function can contribute to abnormal bleeding symptoms.

Platelets are also central to the pathology of cardiovascular disease (CVD), forming thrombi that occlude blood vessels and, hence, cause major adverse cardiovascular events, such as a heart attack (myocardial infarction, MI) and ischemic stroke. For example, thrombi form when platelets aggregate together, and when blood coagulates into a gelatinous form.

More specifically, coronary heart disease (CHD) occurs when the coronary arteries are affected by atherosclerosis, which results in narrowing due to formation of fatty and inflamed plaques within the vessel. Narrowing of these arteries reduces supply of oxygenated blood to the heart and can be further exacerbated by atherosclerotic plaque rupture, which exposes subendothelial matrix proteins such as collagen. These proteins can trigger formation of occlusive thrombi. Platelets play an important role in thrombosis under the high-shear conditions present in atherosclerotic coronary arteries when they respond to collagen and other signs of blood vessel damage by aggregating to form a thrombus. Blood flow can become occluded when a thrombus forms within the coronary artery, resulting in MI. Thrombosis can also occur in other parts of the body including cerebral arteries, the cause of ischaemic strokes, and in veins as well as arteries.

The functions of platelets are also increasingly being linked to other biological processes and pathologies, including cancer (such as cancer metastasis), response to bacterial and viral infection (such as COVID-19), and inflammation (such as chronic inflammatory disease), among others.

Current platelet function tests measure the propensity of platelets to perform functions that are central to haemostasis and thrombosis, such as the formation of aggregates, activation of integrin αllbß3, or the secretion of dense and α-granules. These responses can be triggered by multiple stimuli, such as, but not limited to, collagen, adenosine diphosphate (ADP), and thrombin to which platelets are exposed following vascular damage. There is a large degree of heterogeneity in the way in which platelets respond to these stimuli (Panzer et al., Ann Hematol. 2006;85(2):121-125; Jones et al., J Thromb Haemost. 2007;5(8):1756-1765; Jones C I et al., Blood. 2009;114(7):1405-1416). The majority of current clinical tests for platelet function are highly variable and only useful in certain clinical situations. They also need fresh live cells that have been harvested from the patient in the last few hours, so are of limited use and make large scale screening of platelet function expensive and impractical. There remains a need in the art for a simpler, easier, and reliable method for assessing platelet function.

Anti-platelet medications such as aspirin and clopidogrel can prevent platelet clots and reduce risk of stroke or MI by inhibiting platelet aggregation and reducing the extent of thrombus growth following atherosclerotic plaque rupture. However, anti-platelet medications can also impair haemostasis and increase risk of haemorrhage, including devastating intracerebral brain haemorrhage. Therefore, the risk-to-benefit ratio of anti-platelet medication for patients at risk of cardiovascular events without prior history of CVD does not favour treatment, and is reserved for patients who have already suffered MI. However, patients already receiving anti-platelet therapy following a previous cardiovascular event may still suffer further events (e.g., due to failure of anti-platelet therapy). Accordingly, there remains a need for better identification of individuals most likely to benefit from anti-platelet therapy, so that the benefits and risks of anti-platelet therapy can be more effectively balanced.

### SUMMARY OF THE INVENTION

The present invention relates generally to biomarkers that can be used to assess platelet function. In particular, the invention relates to methods that involve assessing the level of CD36 in platelets of a subject, the level of GPVI in platelets of the subject, and/or the age of the subject as indicators of the platelet function in that subject.

The invention provides a method of assessing platelet reactivity or sensitivity to a platelet activating substance in a sample comprising platelets from a subject, the method comprising:
(i) determining the level of CD36 in the sample;
(ii) determining the level of GPVI in the sample; and/or
(iii) determining the age of the subject from which the platelets were obtained.

The method of assessing platelet reactivity or sensitivity to a platelet activating substance in a sample comprising platelets from a subject may comprise (i) and (ii), (ii) and (iii), (i) and (iii), or (i), (ii) and (iii). A preferred method comprises:
(i) determining a level of CD36 in the sample;
(ii) determining the level of GPVI in the sample; and
(iii) determining the age of the subject.

The invention also provides methods that make use of the method of assessing platelet reactivity or sensitivity to a platelet activating substance to predict the responsiveness of a subject to a therapeutic treatment with an anti-platelet therapy. Accordingly, the invention provides a method of predicting the responsiveness of a subject to a therapeutic treatment with an anti-platelet therapy, the method comprising:
(i) determining the level of CD36 in a sample comprising platelets from the subject;
(ii) determining the level of GPVI in a sample comprising platelets from the subject; and/or
(iii) determining the age of the subject.

This method may comprise any method of assessing platelet reactivity or method of assessing sensitivity to a platelet activating substance in accordance with the present invention.

The methods of the invention may be particularly useful for identifying individuals who may be at increased or decreased risk of, for example, thrombosis, and/or who may be (more) positively or negatively affected by the use of anti-platelet therapy, e.g. at increased or decreased risk of bleeding, e.g., bleeding in response to treatment with an anti-platelet therapy.

### Methods for Identifying Increased Platelet Reactivity

The invention provides methods that make use of the method of assessing platelet reactivity or sensitivity to a platelet activating substance of the invention to determine or identify the presence of increased or high platelet reactivity in a sample. For example, the method of assessing platelet reactivity or sensitivity to a platelet activating substance of the invention may be used to identify a subject having hypersensitive platelets, to identify a subject having high platelet reactivity or sensitivity to a platelet activating substance, to identify a subject with increased risk of a disease, disorder or condition associated with increased platelet reactivity, to identify a subject suitable for treatment with an anti-platelet therapy, or to select a suitable anti-platelet therapy for the subject.

The invention provides a method of identifying a subject having hypersensitive platelets, the method comprising assessing platelet reactivity or sensitivity to a platelet activating substance in a sample comprising platelets from the subject by the method of the invention; wherein the presence of any one, two or three of the following is indicative of the presence of hypersensitive platelets:
(a) a high level of CD36 in the sample;
(b) a high level of GPVI in the sample; and/or
(c) the subject is at least 50 years of age.

The invention also provides a method of identifying a subject having increased or high platelet reactivity or sensitivity to a platelet activating substance, the method comprising assessing platelet reactivity or sensitivity to a platelet activating substance in a sample comprising platelets from the subject by the method of the invention; wherein the presence of any one, two or three of the following is indicative of the presence of increased or high platelet reactivity or sensitivity to a platelet activating substance:
(a) a high level of CD36 in the sample;
(b) a high level of GPVI in the sample; and/or
(c) the subject is at least 50 years of age.

The invention also provides a method of identifying a subject with increased risk of a disease, disorder or condition associated with increased platelet reactivity, such as thrombosis, the method comprising assessing platelet reactivity or sensitivity to a platelet activating substance in a sample comprising platelets from the subject by the method of the invention; wherein the presence of any one, two or three of the following is indicative that the subject has increased risk of a disease, disorder or condition associated with increased platelet reactivity:
(a) a high level of CD36 in the sample;
(b) a high level of GPVI in the sample; and/or
(c) the subject is at least 50 years of age.

The invention also provides a method of identifying a subject suitable for treatment with an anti-platelet therapy, comprising assessing platelet reactivity or sensitivity to a platelet activating substance in a sample comprising platelets from the subject by the method of the invention, wherein the presence of any one, two or three of the following is indicative that the subject is suitable for treatment with an anti-platelet therapy:
(a) a high level of CD36 in the sample;
(b) a high level of GPVI in the sample; and/or
(c) the subject is at least 50 years of age.

The invention also provides a method of identifying a subject with decreased risk of bleeding, such as in response to treatment with an anti-platelet therapy, wherein the presence of any one, two or three of the following is indicative that the subject has a decreased risk of bleeding, wherein the presence of any one, two or three of the following is indicative of a decreased risk of bleeding:
(a) a high level of CD36 in the sample;
(b) a high level of GPVI in the sample; and/or
(c) the subject is at least 50 years of age.

Any of the methods disclosed herein that involve identifying a subject, such as the method of identifying a subject having hypersensitive platelets, the method of identifying a subject having high platelet reactivity or sensitivity to a platelet activating substance, the method of identifying a subject with increased risk of a disease, disorder or condition associated with increased platelet reactivity, the method of identifying a subject suitable for treatment with an anti-platelet therapy, or the method of identifying a subject at decreased risk of bleeding, may further comprise a step of administering an anti-platelet therapy to the subject. For example, such a method may comprise administering an anti-platelet therapy to a subject that has been identified as having hypersensitive platelets, a subject that has been identified as having high platelet reactivity or sensitivity to a platelet activating substance, a subject that has been identified as having increased risk of a disease, disorder or condition associated with increased platelet reactivity, a subject that has been identified as suitable for treatment with an anti-platelet therapy, or a subject that has been identified as at decreased risk of bleeding, by a method as described herein. The anti-platelet therapy may be any suitable therapy, such as any anti-platelet therapy as described herein.

Similarly, the invention provides a method of treating a disease, disorder or condition associated with increased platelet reactivity, the method comprising:
(i) assessing platelet reactivity or sensitivity to a platelet activating substance in a sample comprising platelets from the subject by the method of the invention; and
(ii) administering an anti-platelet therapy to a subject having any one, two or three of the following:
   (a) a high level of CD36;
   (b) a high level of GPVI; and/or
   (c) the subject is at least 50 years of age.

In any of the methods disclosed herein, such as the method of identifying a subject having hypersensitive platelets, the method of identifying a subject having high platelet reactivity or sensitivity to a platelet activating substance, the method of identifying a subject with increased risk of a disease, disorder or condition associated with increased platelet reactivity, the method of identifying a subject suitable for treatment with an anti-platelet therapy, the method of identifying a subject at decreased risk of bleeding, or the method of treating a disease, disorder or condition associated with increased platelet reactivity, the method may determine the presence of any one, two or all three of:
(a) a high level of CD36;
(b) a high level of GPVI; and/or
(c) the subject is at least 50 years of age.

For example, the method may determine the presence of (a) and (b), (b) and (c), (a) and (c), or (a), (b) and (c).

In one embodiment, the subject is at least 50 years of age and the method determines the presence of:
(a) a high level of CD36 in the sample; and
(b) a high level of GPVI in the sample.

In another embodiment, the method determines the presence of all three of:
(a) a high level of CD36 in the sample;
(b) a high level of GPVI in the sample; and
(c) the subject is at least 50 years of age.

Also disclosed herein are the corresponding therapies for use in treatment of a subject having the desired characteristics. The treatment method may comprise carrying out the method of assessing platelet reactivity or sensitivity to a platelet activating substance in a sample comprising platelets from the subject in accordance with the invention, and then treating the subject that has been identified in accordance with the present invention as having increased or high platelet reactivity or sensitivity to a platelet activating substance.

For example, the invention provides an anti-platelet therapy for use in a method of treating a subject in need thereof, the method comprising:
(i) assessing platelet reactivity or sensitivity to a platelet activating substance in a sample comprising platelets from the subject by the method of the invention; and
(ii) administering the anti-platelet therapy to a subject having any one, two or three of the following:
   (a) a high level of CD36 in the sample;
   (b) a high level of GPVI in the sample; and/or
   (c) the subject is at least 50 years of age.

The invention also provides an anti-platelet therapy for use in a method of treating a subject that has the characteristic(s) of increased or high platelet reactivity or sensitivity to a platelet activating substance as described herein. For example, the invention provides an anti-platelet therapy for use in a method of treating a subject in need thereof, wherein the subject has:
(a) a high level of CD36 in their platelets;
(b) a high level of GPVI in their platelets; and/or
(c) an age of at least 50 years.

In such a use, or in the corresponding method of treatment, which also forms a part of the present invention, the method of assessing platelet reactivity or sensitivity to a platelet activating substance of the present invention may or may not be performed as a part of the use or method of treatment.

The subject may have (a) and (b), (b) and (c), (a) and (c), or (a), (b) and (c). For example, in some embodiments, the subject has all three of:
(a) a high level of CD36;
(b) a high level of GPVI; and
(c) an age of at least 50 years.

Where the invention relates to treating a subject, the treatment may be treatment of a disease, disorder or condition associated with increased platelet reactivity. The disease, disorder or condition associated with increased platelet reactivity may be selected from: a cardiovascular disease, a metabolic or endocrine disease, an abnormal blood condition, an abnormal platelet count, a cancer, a response to a bacterial and/or a viral infection, and/or inflammation. The cardiovascular disease may be selected from the group consisting of a thrombosis, a myocardial infarction, a stroke, a peripheral arterial disease, an aortic disease, and/or a coronary heart disease.

### Methods for Identifying Decreased Platelet Reactivity

The invention also provides methods that make use of the method of assessing platelet reactivity or sensitivity to a platelet activating substance of the invention to determine or identify the presence of decreased or low platelet reactivity in a sample. For example, the method of assessing platelet reactivity or sensitivity to a platelet activating substance of the invention may be used to identify a subject having hyposensitive platelets, to identify a subject having low platelet reactivity or sensitivity to a platelet activating substance, to identify a subject with decreased risk of a disease, disorder or condition associated with increased platelet reactivity, to identify a subject not suitable for treatment with an anti-platelet therapy, or to identify a subject at increased risk of bleeding.

For example, the invention provides a method of identifying a subject having hyposensitive platelets, the method comprising assessing platelet reactivity or sensitivity to a platelet activating substance in a sample comprising platelets from the subject by the method of the invention, wherein the presence of any one, two or three of the following is indicative of the presence of hyposensitive platelets:
(a) a low level of CD36 in the sample;
(b) a low level of GPVI in the sample; and/or
(c) the subject is less than 50 years of age.

The invention also provides a method of identifying a subject with decreased risk of a disease, disorder or condition associated with increased platelet reactivity, such as thrombosis, the method comprising assessing platelet reactivity or sensitivity to a platelet activating substance in a sample comprising platelets from the subject by the method of the invention; wherein the presence of any one, two or three of the following is indicative that the subject has decreased risk of a disease, disorder or condition associated with increased platelet reactivity:
(a) a low level of CD36 in the sample;
(b) a low level of GPVI in the sample; and/or
(c) the subject is less than 50 years of age.

The invention provides a method of identifying a subject not suitable for treatment with an anti-platelet therapy, the method comprising assessing platelet reactivity or sensitivity to a platelet activating substance in a sample comprising platelets from the subject by the method of the invention, wherein the presence of any one, two or three of the following is indicative that the subject may be unsuitable for treatment with anti-platelet therapy:
(a) a low level of CD36 in the sample;
(b) a low level of GPVI in the sample; and/or
(c) the subject is less than 50 years of age.

The invention provides a method of identifying a subject at decreased risk of bleeding, such as in response to treatment with an anti-platelet therapy, the method comprising assessing platelet reactivity or sensitivity to a platelet activating substance in a sample comprising platelets from the subject by the method of the invention, wherein the presence of any one, two or three of the following is indicative that the subject is at decreased risk of bleeding:
(a) a low level of CD36 in the sample;
(b) a low level of GPVI in the sample; and/or
(c) the subject is less than 50 years of age.

In any of the methods disclosed herein, such as the method of identifying a subject having hyposensitive platelets, the method of identifying a subject having low platelet reactivity or sensitivity to a platelet activating substance, the method of identifying a subject with decreased risk of a disease, disorder or condition associated with increased platelet reactivity, the method of identifying a subject not suitable for treatment with an anti-platelet therapy, the method of identifying a subject at increased risk of bleeding, or the method of treating a disease, disorder or condition associated with decreased or low platelet reactivity, the method may determine the presence of any one, two or all three of:
(a) a low level of CD36;
(b) a low level of GPVI; and/or
(c) the subject is less than 50 years of age.

For example, the method may determine the presence of (a) and (b), (b) and (c), (a) and (c), or (a), (b) and (c). In some embodiments, the method determines the presence of all three of:
(a) a low level of CD36 in the sample;
(b) a low level of GPVI in the sample; and
(c) the subject is less than 50 years of age.

In some embodiments, the subject is less than 50 years of age and the method determines the presence of:
(a) a low level of CD36 in the sample; and
(b) a low level of GPVI in the sample.

### Biomarkers

The invention relates to the use of one, two or three biomarkers to assess platelet reactivity.

One of the biomarkers that may be used in accordance with the present invention is the age of the subject.

As disclosed herein, the presence of increased or high platelet reactivity may be associated with a subject having a greater age, such as an age of at least 50 or at least 55 years of age.

Accordingly, a method of the invention that determines the presence of increased or high platelet reactivity may be performed on a subject, or a sample from a subject, wherein the subject is at least years of age; or the subject is at least 55 years of age.

As disclosed herein, the presence of decreased or low platelet reactivity may be associated with a subject having a lower age, such as an age of less than 50 or less than 45 years of age.

Accordingly, a method of the invention that determines the presence of decreased or low platelet reactivity may be performed on a subject, or a sample from a subject, wherein the subject is less than 50 years of age; or the subject is less than 45 years of age.

Another biomarker that may be used in accordance with the present invention is the level of CD36 in platelets or in a sample comprising platelets.

As disclosed herein, a high level of CD36 may be identified as:
(i) a level of CD36 that is in the top 35^{th} percentile compared to a reference population;
(ii) a level of CD36 that is at least 15% higher than the level of CD36 in a control or reference sample comprising platelets; or
(iii) a platelet or a population of platelets expressing at least 8000 copies of CD36 per platelet.

As disclosed herein, a low level of CD36 may be identified as:
(i) a level of CD36 that is in the bottom 65^{th} percentile compared to a reference population;
(ii) a level of CD36 that is at least 15% lower than the level of CD36 in a control or reference sample comprising platelets; or
(iii) a platelet or a population of platelets expressing fewer than 8000 copies of CD36 per platelet.

Another biomarker that may be used in accordance with the present invention is the level of GPVI in platelets or in a sample comprising platelets.

As disclosed herein, a high level of GPVI may be identified as:
(i) a level of GPVI that is in the top 50^{th} percentile compared to a reference population;
(ii) a level of GPVI that is at least 15% higher than the level of GPVI in a control or reference sample comprising platelets; or
(iii) a platelet or a population of platelets expressing at least 3000 copies of GPVI per platelet.

As disclosed herein, a low level of GPVI may be identified as:
(i) a level of GPVI that is in the bottom 50^{th} percentile compared to a reference population;
(ii) a level of GPVI that is at least 15% lower than the level of GPVI in a control or reference sample comprising platelets; or
(iii) a platelet or a population of platelets expressing fewer than 3000 copies of GPVI per platelet.

### Other Aspects

Methods of the invention may be performed on a sample. The sample should comprise platelets, such as a sample comprising platelets from a subject. In some embodiments, the sample: (i) consists of platelets from the subject; (ii) comprises or consists of a population of platelets from the subject; (iii) is a cell-free sample; (iv) is whole blood; or (v) is plasma.

In some embodiments, the sample is from a subject that has not received anti-platelet therapy.

In some embodiments, the subject has no previous history of a disease, disorder or condition associated with increased platelet reactivity, optionally selected from: a cardiovascular disease, a metabolic or endocrine disease, an abnormal blood condition, an abnormal platelet count, a cancer, a response to a bacterial and/or a viral infection, and/or inflammation. For example, in some embodiments the subject has no previous history of cardiovascular disease.

In some embodiments, the subject has, or is at risk of, a disease, disorder or condition selected from the following: a cardiovascular disease, a metabolic or endocrine disease, an abnormal blood condition, an abnormal platelet count, a cancer, a response to a bacterial and/or a viral infection, and/or inflammation. In some embodiments, the subject is, or will be: receiving blood thinning medication, undergoing surgery, and/or a blood donor.

Where the invention pertains to a disease, disorder or condition associated with increased platelet reactivity, then in some embodiments that disease, disorder or condition is selected from: a cardiovascular disease, a metabolic or endocrine disease, an abnormal blood condition, an abnormal platelet count, a cancer, a response to a bacterial and/or a viral infection, and/or inflammation. In some embodiments the cardiovascular disease is selected from the group consisting of a thrombosis, a myocardial infarction, a stroke, a peripheral arterial disease, an aortic disease, and/or a coronary heart disease.

Where the invention pertains to an anti-platelet therapy, in some embodiments, the anti-platelet therapy is a platelet agglutination inhibitor or a platelet aggregation inhibitor. In some embodiments, the anti-platelet therapy is selected from the group consisting of: aspirin,, dipyridamole, cyclooxygenase inhibitors (for example, masoprocol, indomethacin, nabumetone, celecoxib, tolmetin, rofecoxib, piroxicam, fenoprofen, valdecoxib, and/or diclofenac), ADP receptor (P2Y) antagonists (for example, clopidogrel, prasugrel, ticagrelor, and/or cangrelor), GPVI inhibitors (for example, glenzocimab, revacept, honokiol, losartan, and/or tussilagone), a drug that targets another platelet receptor, such as a GPIIbIlla inhibitor (for example, tirofiban and/or eptifibatide), and/or a non-steroidal anti-inflammatory drug (NSAID).

In some embodiments, the invention comprises determining the level of CD36 and/or GPVI in a sample. In some embodiments, the level of CD36 and/or the level of GPVI is determined by: (i) an ELISA; (ii) a lateral flow test; (iii) flow cytometry; (iv) an immunological assay; (v) an antibody assay; (vi) a two-site antibody assay; or (vii) quantitative mass spectrometry.

### BRIEF DESCRIPTION OF THE FIGURES

Embodiments of the invention are now described, by way of example only, with reference to the accompanying drawings:

FIG. 1. Thrombus formation on type I collagen is highly variable and correlates with sensitivity to stimulation with platelet activating substances

A. Schematic illustration of thrombus formation measured in vitro

Thrombus formation can be measured in vitro by perfusing human whole blood over type I collagen and imaging incorporation of labelled platelets into thrombi using confocal fluorescence microscopy

B. Variation in thrombus formation between individuals

Bi. Graph of thrombus growth (arbitrary fluorescence units (AFU)) measured in real time for 27 healthy donors.

Bii. Size of thrombi after 520 s.

C. Schematic illustration of receptors stimulated by ADP, TRAP-6 and CRP-XL in the PPAnalysis assay

An analysis technique (Platelet Phenomic Analysis or PPAnalysis) was developed to help understand the differences in the way different people's platelets make clots when stimulated by three different compounds: ADP, TRAP-6 and CRP-XL. Includes functional output of α-granule release measured via exposure of p-selectin on the platelet surface

D. Two different types of measurement are obtained by PPAnalysis: sensitivity and capacity

Representation of a concentration-response curve used to extract sensitivity and capacity metrics from the PPAnalysis assay data.

E. Sensitivity (Ei) and capacity (Eii) outputs measured using PPAnalysis in a cohort of 106 healthy donors

When the sensitivity and capacity measured in response to the three compounds (ADP, TRAP-6, CRP-XL) are compared to the thrombus formation measurements, high sensitivity to CRP-XL is linked to formation of larger thrombi.

F. Comparison of sensitivity and capacity outputs

Scatter plots with linear regression comparing sensitivity and capacity outputs for ADP, TRAP-6 and CRP-XL in a subset of 23 blood samples for which thrombus formation was also measured. r² and p values were calculated using Pearson correlation.

FIG. 2. Sensitivity of GPVI responses can be predicted from age and platelet surface expression of GPVI and CD36

A. Dot plots of Ai) sensitivity and Aii) capacity for a cohorts of 461 healthy donors

A second larger cohort of blood donors were recruited to investigate determinants of the sensitivity to platelet activating substances by performing PPAnalysis.

B. Normalised levels of panel of platelet surface proteins measured by flow cytometry

Measuring the amount of several different proteins on the surface of platelets that might be a source of variability of sensitivity to platelet activating substances.

C. Recording the age, platelet count, mean platelet volume (MPV in femtolitres, fL) and BMI (body mass index) of the donors

F represents female donors, M represents male donors.

D. The data was used to make a model to predict sensitivity to platelet activating substances

The cohort was split into training and test cohorts-one to generate the model and the other one to test it on. The cohort was randomly assigned to training and test cohorts at 7:3 ratio. Stepwise regression with stepwise selection was performed using training cohort data to identify predictors of sensitivity to platelet activating substances.

E. A model was made to identify parameters that could predict the sensitivity to platelet activating substances

The model identified three variables; GPVI expression, CD36 expression and age as the combination with the highest r² and lowest Route Mean Square Error (RMSE) and Mean Absolute Error (MAE). These three parameters gave the best prediction of sensitivity to platelet activating substances.

F. Each of these parameters (GPVI expression, CD36 expression and age) were tested on the test cohort

Scatter plots of Fi) GPVI expression, Fii) CD36 expression and Fiii) age plotted against sensitivity to CRP-XL measured in the test cohort confirmed that these associations were also present in the test cohort. Fiv) Scatter plot comparing the sensitivity to CRP-XL predicted for the test cohort using the multiple regression parameters identified using the training cohort data. r² and p values were calculated using Pearson's correlation. Each parameter was individually associated with sensitivity to CRP-XL and when the three parameters were used together they could predict sensitivity to CRP-XL more accurately then when they were used on their own.

FIG. 3. CD36 is a strong predictor of sensitivity to platelet activating substances in the over 55 s

A. Scatter plots and linear regression for sensitivity to CRP-XL against CD36 expression in healthy donors <55 (Ai) or ≥55 (Aii) years old

r² and p values were calculated using Pearson correlations. CD36 is better as predicting sensitivity to CRP-XL in older people (over 55 years).

B. Scatter plots and linear regression for sensitivity to CRP-XL against GPVI expression in healthy donors <55 (Bi) or ≥55 (Bii) years old r² and p values were calculated using Pearson correlations. GPVI is equally good at predicting sensitivity to CRP-XL in people over and under 55 years.

C. ROC plots for CD36 and GPVI expression as predictors for high (top 33%) sensitivity to CRP-XL for healthy donors <55 (Ci) or ≥55 (Cii) years old

CD36 as good as GPVI for prediction of sensitivity to CRP-XL in the over 55 s, but worse in the under 55 s.

FIG. 4. Stratification of sensitivity to platelet activating substances using age and expression of GPVI and CD36

A. Stratification algorithm to predict high, medium or low sensitivity to platelet activating substances on the basis of age (55 years or over), CD36 expression (high=top 33%) and GPVI expression (high=top 50%)

An algorithm was created to predict if a donor was likely to have high, medium or low sensitivity to platelet activating substances based on age, CD36 and GPVI levels.

B. Plot of sensitivity to CRP-XL for donors after stratification into low, medium and high subpopulations

The algorithm could separate donors on the basis of sensitivity to CRP-XL. Bars are the mean±S.D. and p values were calculated using 1-way ANOVA with Tukey multiple comparisons test.

FIG. 5. Three-factor stratification predicts sensitivity to platelet activating substances of patients with ischaemic heart disease

A. Scatter plot of sensitivity to CRP-XL compared to GPVI expression

A cohort of 34 patients with ischaemic heart disease were recruited and it was found that sensitivity to CRP-XL could be predicted by GPVI levels. r² and p values were calculated using Pearson correlation.

B. Scatter plot of sensitivity to CRP-XL compared to CD36 expression

In the same cohort, sensitivity to CRP-XL could be predicted by CD36 levels. r² and p values were calculated using Pearson correlation.

C. Scatter plot of sensitivity to CRP-XL compared to age

In the same cohort, sensitivity to CRP-XL could not be predicted by age-possibly because some patients were taking antiplatelet medication (aspirin). r² and p values were calculated using Pearson correlation.

D. Plot comparing the age of patients free from or receiving aspirin (ASA)

The age of patients taking aspirin was compared to those not taking aspirin and found that patient taking aspirin were older on average. p values were calculated using student's t-tests.

E. Plot comparing the sensitivity CRP-XL of patients free from or receiving aspirin (ASA)

The patients taking aspirin also had lower sensitivity to CRP-XL-suggesting this is why the link between age and sensitivity to CRP-XL was not present in this cohort of patients. p values were calculated using student's t-tests.

F. Plot of sensitivity to CRP-XL for patients stratified into groups predicted to have low, medium or high sensitivity for CRP-XL

When the algorithm (FIG. 4) was used to predict high, medium and low sensitivity to CRP-XL, it was found that few patients in this cohort were predicted to have low sensitivity (due to older age of patients) but patients predicted to have high sensitivity were more sensitive to CRP-XL than those predicted to have medium sensitivity. p values were calculated using 1-way ANOVA with Bonferroni multiple comparisons test.

FIG. 6. Three-factor stratification predicts thrombus formation of patients with ischaemic heart disease

A. Scatter plot of in vitro thrombus volume compared to CRP-XL sensitivity

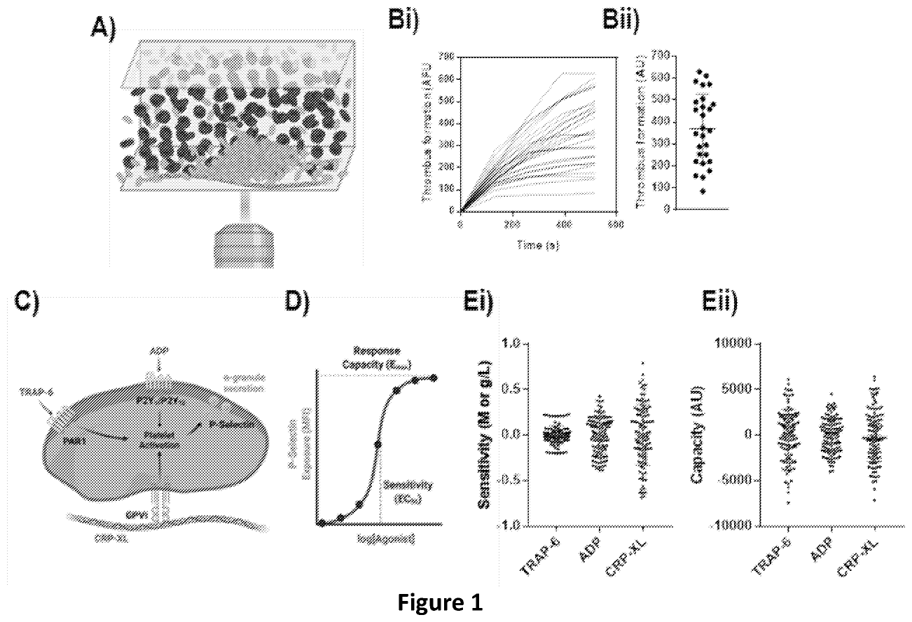

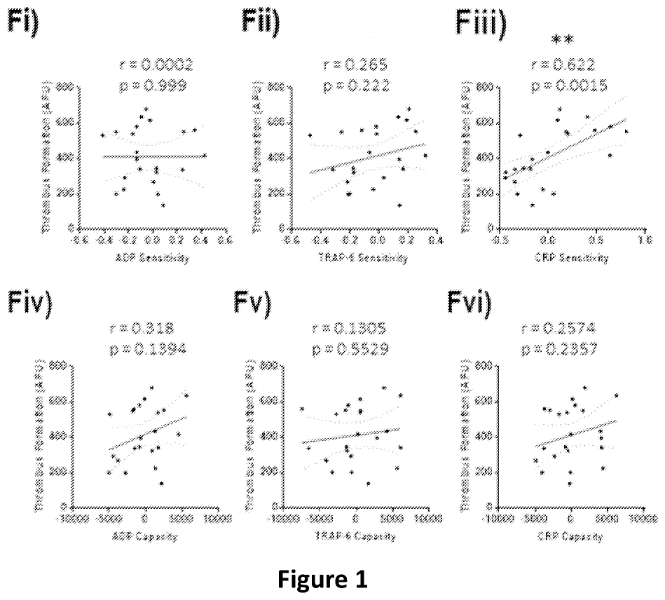

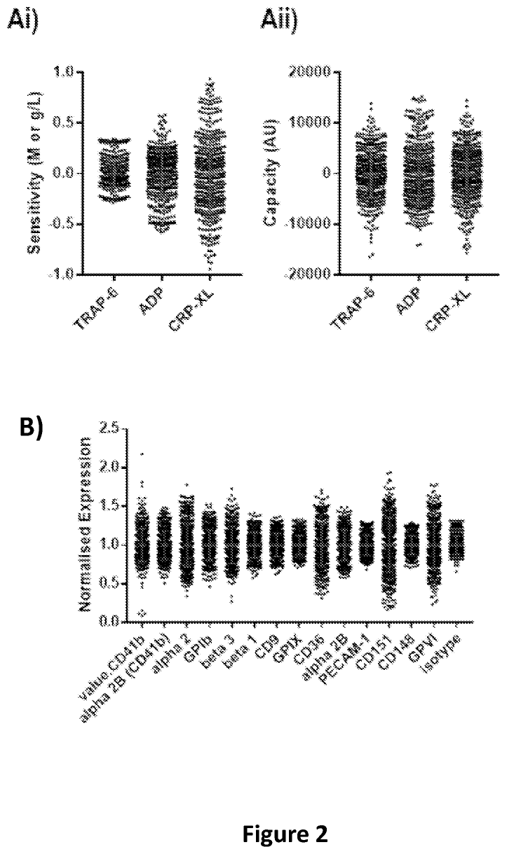

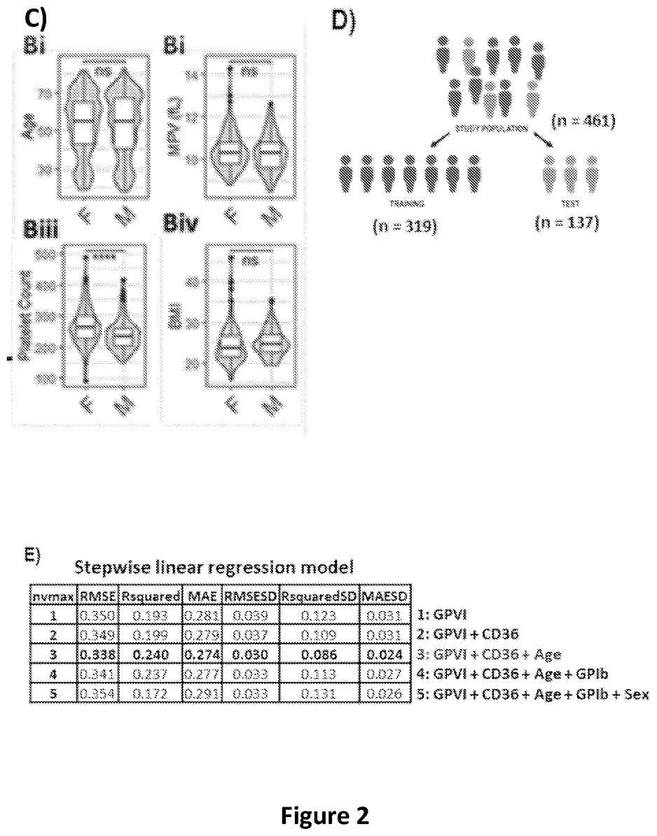

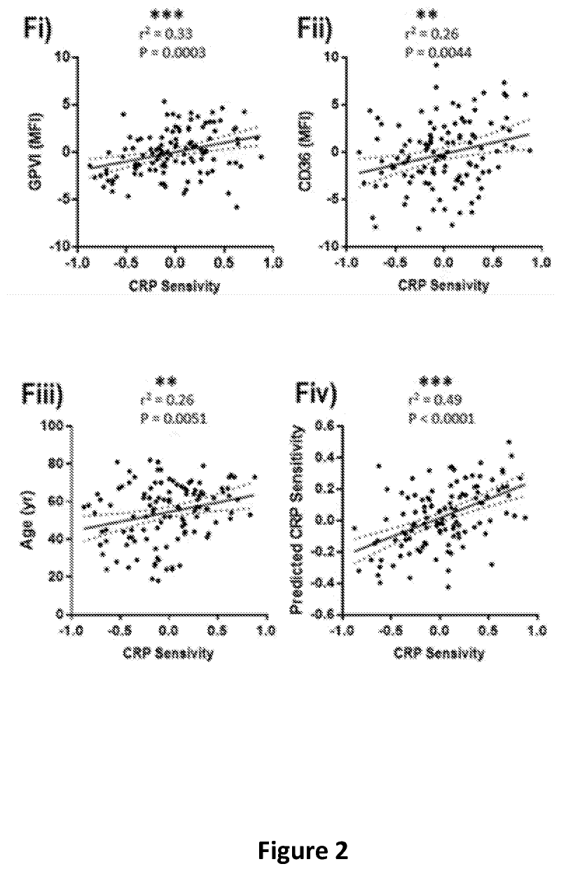

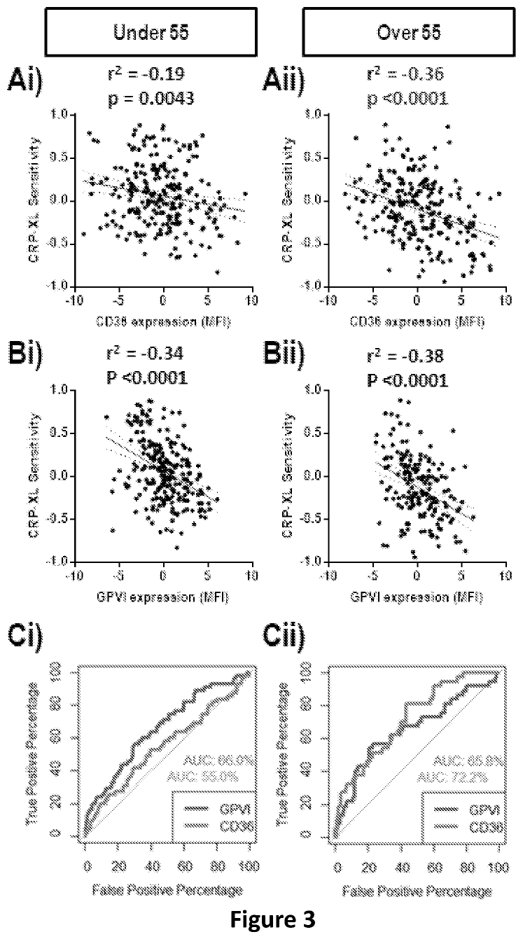

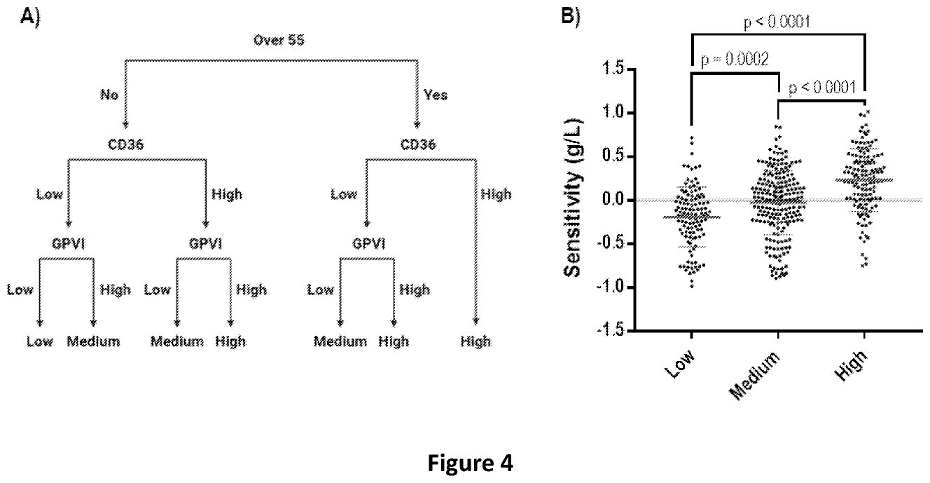

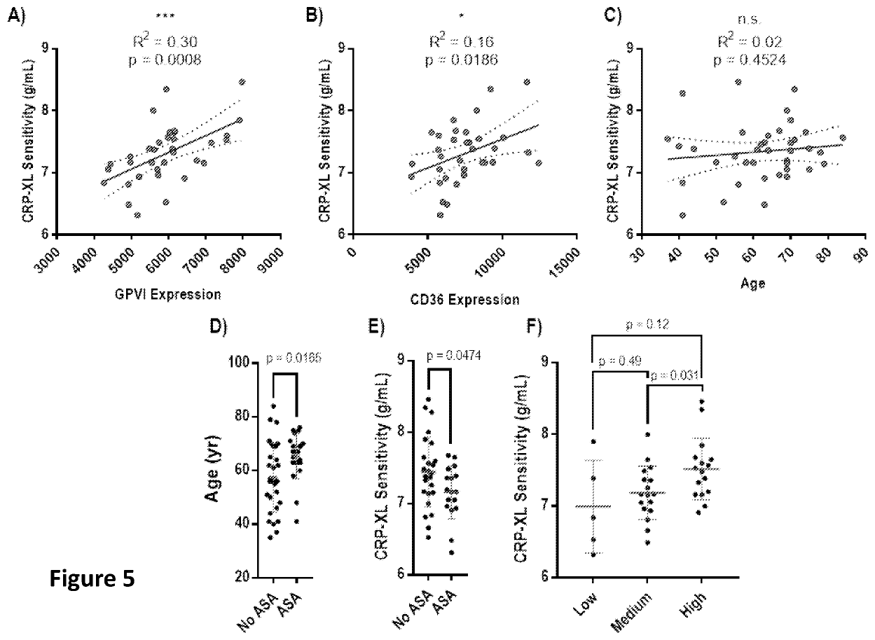

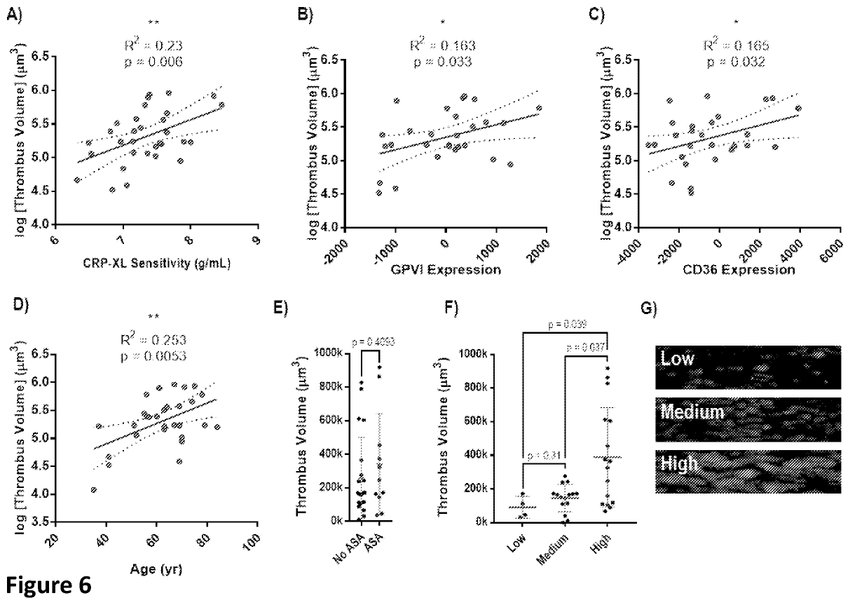

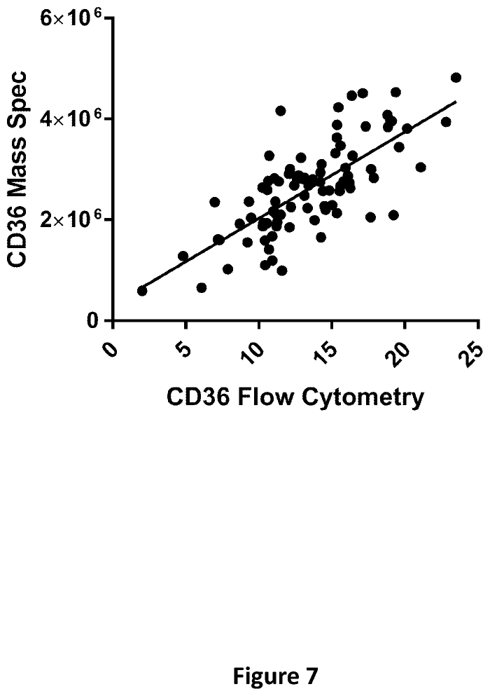

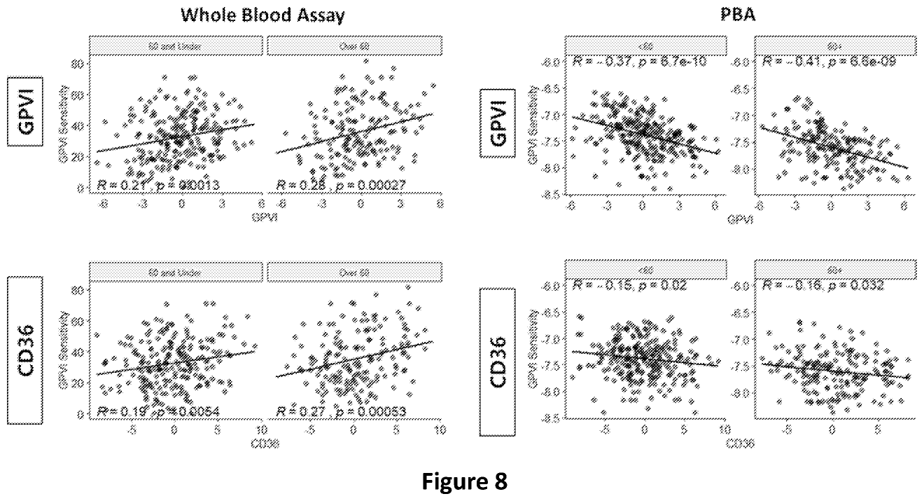

Thrombus formation in vitro was also measured in the cohort of 34 patients with ischaemic heart disease. Thrombus formation was linked to sensitivity to CRP-XL (just as it was in FIG. 1F). r² and p values were calculated using Pearson correlation.

Scatter plot of in vitro thrombus volume compared to GPVI expression

Thrombus formation in vitro was also measured in the cohort of 34 patients with ischaemic heart disease. Thrombus formation was linked to GPVI levels. r² and p values were calculated using Pearson correlation.

B. Scatter plot of in vitro thrombus volume compared to CD36 expression

Thrombus formation in vitro was also measured in the cohort of 34 patients with ischaemic heart disease. Thrombus formation was linked to CD36 levels. r² and p values were calculated using Pearson correlation.

C. Scatter plot of in vitro thrombus volume compared to age

Thrombus formation in vitro was also measured in the cohort of 34 patients with ischaemic heart disease. Thrombus formation was linked to age (which was surprising as previously found that sensitivity to CRP-XL was not linked to age due to aspirin use by older patients). r² and p values were calculated using Pearson correlation.

D. Plots comparing the in vitro thrombus volumes of patients free from or receiving aspirin (ASA)

The link between thrombus formation and age was present because thrombus formation was not reduced in patients that were taking aspirin. p values were calculated using student's t-tests.

E. Plot of in vitro thrombus volumes for patients stratified into groups predicted to have low, medium or high sensitivity to platelet activating substances

Patients stratified into groups predicted to have low medium and high sensitivity groups also had different levels of thrombus formation and the patients with largest thrombi were all in the group predicted to have high sensitivity to platelet activating substances, p values were calculated using 1-way ANOVA with Bonferroni multiple comparisons test.

F. Representative images from in vitro thrombus formation experiments for patients predicted to have low, medium or high sensitivity to platelet activating substances

FIG. 7. CD36 flow cytometry vs CD36 mass spectrometry

FIG. 8. Other measures (whole blood fluorescence-activated cell sorting (FACS) assay (WBA) and plate-based aggregation (PBA)) of platelet functional responses regressed against CD36 and GPVI expression

### DETAILED DESCRIPTION

The inventors have surprisingly found a set of markers that can be used to assess platelet sensitivity or platelet reactivity. For example, the levels of CD36 and GPVI in a population or sample of platelets, and the age of the subject from whom the sample was obtained, have been found to be linked to sensitivity to platelet activating substances such as collagen-related peptide (CRP, a GPVI receptor activator for platelet activation).

These markers were identified using platelets from healthy volunteers and the present invention arose from complex analyses of platelet function, including machine learning approaches to analyse complex combinations of multiple parameters. The identity of these markers was established using extensive analysis of healthy and diseased subjects' platelets using a high throughput and ‘big data’ approach to platelet cell function analysis in clinical or pharmaceutical studies. Al-based analysis technology and complex statistical analysis were used to reveal the unique combination of biomarkers that correlate with platelet functional reactivity. The validity of these as biomarkers of platelet function was replicated in the whole 500-strong cohort, and in a small cohort of cardiovascular disease patients.

The markers enable the prediction of platelet function without requiring platelet function itself to be measured. This renders investigation of platelet reactivity in large cohorts feasible and enables incorporation of reactivity into MI risk treatment algorithms, amongst other applications of the invention. This enables platelet reactivity to be assessed without the need to actually measure platelet function, which is specialised in nature, requires analysis immediately after a blood sample is taken and requires specialist equipment. The methods of the present invention may also be used to determine disease risk or appropriate therapies for non-thrombotic conditions including cancer, response to bacterial and viral infection, and inflammation among others.

Accordingly, the present invention pertains to markers that enable identification of subjects with hypersensitive platelets that could benefit from personalised anti-platelet therapy to reduce the risk of cardiovascular events-which may include heart attacks and strokes-amongst other platelet-associated conditions. Previous attempts to stratify which subjects will benefit from anti-platelet drugs on basis of patient characteristics (such as age, disease status, or body mass index (BMI)) or platelet function have been generally unsuccessful.

The markers can also be used to identify subjects likely to experience failure of therapy due to platelet hypersensitivity. Such subjects may benefit from altered therapy, such as therapy targeting their hypersensitive platelets. For example, an altered therapy may include treatment with an anti-platelet therapy, or treatment with an altered, e.g., increased, dose of anti-platelet therapy. For example, the markers may be used to identify subjects at increased risk of thrombosis due to platelet hypersensitivity. This may be used to select those subjects for treatment with anti-thrombotic therapy such as anti-platelet therapy or anticoagulant therapy. The markers can also be used to identify subjects who may be at decreased risk of bleeding, e.g., bleeding in response to anti-platelet therapy. Such subjects may benefit from altered therapy, such as an altered, e.g., increased, dose of anti-platelet therapy.

The markers can also be used to identify subjects with hyposensitive platelets. Such subjects may be less likely to respond to anti-platelet therapy. Such subjects may benefit from altered therapy, such as therapy targeting their hyposensitive platelets. For example, this may include an altered, e.g., reduced, dose of anti-platelet therapy, or treatment with a platelet agonist or clotting agent. The markers may be used to identify subjects at decreased risk of thrombosis due to platelet hyposensitivity. This may be used to select those subjects not requiring treatment with anti-thrombotic therapy such as anti-platelet therapy. The markers can also be used to identify subjects who may be at increased risk of bleeding, e.g., bleeding in response to anti-platelet therapy, due to their platelets being hyposensitive. Such subjects may benefit from altered therapy, such as an altered, e.g., decreased, dose of anti-platelet therapy to reduce or minimise bleeding, or administration of therapy intended to prevent or reduce bleeding, such as a platelet agonist or clotting agent.

In contrast to platelet function tests, which are expensive and require specialist equipment not present in most hospitals, measurement of biomarkers in accordance with the present invention can be standardised and performed using generic equipment.

Thus, key benefits of the invention include:
prevention of unnecessary treatment with anti-platelet drugs which can cause side effects such as haemorrhage;
the provision of an alternative to platelet function analysis which is typically specialised, and time sensitive;

the ability to use simple, routine and inexpensive tests to determine the relevant protein biomarker levels;
the ability to assess platelet function by testing stored samples;

the ability to run smaller, more targeted and/or cheaper clinical trials for new treatments. The invention will be useful to patients on blood thinning drugs, undergoing major surgery, with abnormal blood conditions or platelet counts, to blood donors, or in other circumstances where bleeding or clotting might be an issue.

Unless defined otherwise herein, all technical and scientific terms used herein have the same meaning as commonly understood by one of ordinary skill in the art to which the invention pertains. It should be understood that this invention is not limited to the particular methodology, protocols, and reagents, etc., described herein and as such can vary. The terminology used herein is for the purpose of describing particular instances only, and is not intended to limit the scope of the present invention, which is defined solely by the claims.

The term “a” and “an” refers to one or to more than one (i.e., to at least one) of the grammatical object of the article. By way of example, “an element” means one element or more than one element.

The term “about” when referring to a measurable value such as an amount, a temporal duration, and the like, is meant to encompass variations of ±20% or in some instances ±10%, or in some instances ±5%, or in some instances ±1 %, or in some instances ±0.1 % from the specified value, as such variations are appropriate to perform the disclosed methods.

The term “subject” is intended to include living organisms in which an immune response can be elicited. Preferably, the subject is a mammal. The methods described herein may be useful for human or veterinary applications. More preferably, the subject is a human. In one embodiment, the subject is a patient, e.g., a subject in need of treatment, or a subject that is receiving or is about to receive treatment. The subject may be an infant, a child or an adult. Preferably the subject is a human adult. The subject may be a human and may be at least 20 years old, at least 30 years old, at least 40 years old, at least 50 years old, at least 60 years old, at least 70 years old, or at least 75 years old.

Throughout this disclosure, various embodiments of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99% identity, includes something with 95%, 96%, 97%, 98%, or 99% identity, and includes subranges such as 96-99%, 96-98%, 96-97%, 97-99%, 97-98%, and 98-99% identity. This applies regardless of the breadth of the range.

Various embodiments of the compositions and methods herein are described in further detail below. Additional definitions are set out throughout the specification.

### Biomarkers

Methods described herein involve assessing one or more biomarkers, such as CD36 and/or GPVI, in a sample comprising platelets from a subject, and/or determining the age of the subject from which the platelets were obtained.

The term “biomarker” refers to a biological marker which indicates a particular physiological or pathological state, process, response, disease, disorder or condition. A marker may be a physical marker such as a naturally occurring molecule (e.g., a DNA molecule, a gene, an allele, an RNA molecule, or a protein), or may be a characteristic (e.g., age) that can be measured. The terms “biomarker” and “marker” are used interchangeably herein. In the context of the present invention, the marker may be CD36. In the context of the present invention, the marker may be GPVI. In the context of the present invention, the age of the subject may be a marker.

The term “CD36” refers to the glycoprotein cluster of differentiation 36 (CD36) receptor, also known as platelet glycoprotein 4, fatty acid translocase (FAT), scavenger receptor class B member 3 (SCARB3), and glycoproteins 88 (GP88), IIIb (GPIIIB), or IV (GPIV). The CD36 antigen is an integral membrane protein, and is present on the surface of platelets.

The term “GPVI” refers to glycoprotein VI, a glycoprotein receptor for collagen that is present on the surface of platelets. GPVI is also known as GP6, BDPLT11, GPIV, and glycoprotein VI platelet.

The methods of the invention may comprise a step of determining the age of the subject. Such determining may involve obtaining data from a patient record or from the subject. In the methods of the invention the step of determining the age of the subject may be omitted. For example, in some embodiments the age of the subject is known or already recorded, and so it may not be necessary to determine the age of that subject. For example, in some embodiments the methods may be carried out on a sample from a subject of known age. The age of the subject is preferably expressed in whole years, so that for example a subject who is greater than 50 years of age but less than 51 years of age is classified as having an age of 50.

The term “level” in relation to a biomarker such as CD36 or GPVI may refer to an amount of the biomarker protein or a nucleic acid molecule encoding the biomarker protein. In some methods of the invention, a level of a biomarker protein is evaluated. In some methods, a level of a nucleic acid molecule encoding a biomarker protein is evaluated. In some methods, an mRNA molecule encoding a biomarker protein is evaluated. In some methods, a DNA molecule encoding a biomarker protein is evaluated. In some methods of the invention, an expression level of a biomarker is evaluated. In some methods of the invention, an amount such as a quantified amount or relative amount or a copy number or relative copy number of a biomarker is evaluated. The amount may be assessed by directly measuring the amount of the relevant molecule, or may be assessed indirectly by assessing binding of the molecule to a known ligand or by assessing the function of the relevant molecule. For example, the level of a marker may be assessed using binding of the marker to a specific binding partner such as an antibody or other binding molecule that is specific for that marker. In the methods of the present invention, the biomarker may be CD36 and/or GPVI. In the methods of the invention, the level of CD36 and/or GPVI may be determined. In the methods of the invention, the expression level of CD36 and/or GPVI may be assessed. In the methods of the invention, the amount of CD36 protein and/or GPVI protein may be assessed. In some embodiments, the level or amount of CD36 and/or GPVI is determined by: an ELISA, a lateral flow test, flow cytometry, an immunological assay, an antibody assay, a two-site antibody assay, or quantitative mass spectrometry.

The term “expression” refers to the transcription and/or translation of a particular nucleotide sequence. In some embodiments, expression comprises translation of an mRNA introduced into a cell. Unless otherwise noted, classifying a cell, a population of cells, a platelet, a population of platelets, or a sample or composition comprising cells and/or platelets, as “not expressing,” or having an “absence of” or being “negative for” a particular marker may not necessarily mean an absolute absence of the marker. The skilled artisan can readily compare the cell, platelet, population, sample or composition against a positive and/or a negative control, and/or set a predetermined threshold, and classify the cell, platelet, population, sample or composition as not expressing or being negative for the marker when the cell, platelet, population, sample or composition has an expression level below the predetermined threshold or has an overall expression level below the predetermined threshold using conventional detection methods, e.g., using flow cytometry, for example.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (for example, rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “homologous” or “identity” refers to the subunit sequence identity between two polymeric molecules, for example, between two nucleic acid molecules, such as, two DNA molecules or two RNA molecules, or between two polypeptide molecules. When a subunit position in both of the two molecules is occupied by the same monomeric subunit; for example, if a position in each of two DNA molecules is occupied by adenine, then they are homologous or identical at that position. The homology between two sequences is a direct function of the number of matching or homologous positions; for example, if half (for example, five positions in a polymer ten subunits in length) of the positions in two sequences are homologous, the two sequences are 50% homologous; if 90% of the positions (for example, 9 of 10), are matched or homologous, the two sequences are 90% homologous.

The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “nucleic acid,” “nucleic acid molecule,” “polynucleotide,” or “polynucleotide molecule” refers to deoxyribonucleic acids (DNA) or ribonucleic acids (RNA) and polymers thereof in either single-or double-stranded form. Unless specifically limited, the term encompasses nucleic acids containing known analogues of natural nucleotides that have similar binding properties as the reference nucleic acid and are metabolized in a manner similar to naturally occurring nucleotides. In some embodiments, a “nucleic acid,” “nucleic acid molecule,” “polynucleotide,” or “polynucleotide molecule” comprise a nucleotide/nucleoside derivative or analog. Unless otherwise indicated, a particular nucleic acid sequence also implicitly encompasses conservatively modified variants thereof (for example, degenerate codon substitutions, for example, conservative substitutions), alleles, orthologs, SNPs, and complementary sequences as well as the sequence explicitly indicated. Specifically, degenerate codon substitutions, for example, conservative substitutions may be achieved by generating sequences in which the third position of one or more selected (or all) codons is substituted with mixed-base and/or deoxyinosine residues (Batzer et al., Nucleic Acid Res. 19:5081(1991 ); Ohtsuka et al., J. Biol. Chem. 260:2605-2608 (1985); and Rossolini et al., Mol. Cell. Probes 8:91-98 (1994)).

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types.

“Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

The invention may utilise antibodies. The antibody may bind specifically to its intended target, such as CD36 or GPVI, for example it may bind to that target and not to other related molecules. For example, the antibody may bind to CD36 or GPVI but not other naturally occurring glycoprotein receptors. The antibody may bind to CD36 or GPVI but not other naturally occurring molecules on the surface of platelets. The antibody may bind to human CD36 or human GPVI but not to CD36 or GPVI from a different species. The antibody may be a monoclonal or polyclonal antibody. The antibody may be a humanized antibody. The antibody may be a human antibody. The term “antibody” also includes fragments of a full antibody that retain binding to the relevant target antigen. Suitable antigen-binding fragments may include Fab, Fc, Fv, scFv, dAb, Fd, Fab′, F(ab′)2 or an isolated complementarity determining region (CDR) having sufficient framework to bind the target. The antibody may not have an Fc region. A Fab fragment may be a monovalent fragment consisting of the VL, VH, CL and CH1 domains. A F(ab′)2 fragment may be a bivalent fragment comprising two Fab fragments linked by a disulfide bridge at the hinge region. A Fc fragment may consist of the CH2 and CH3 domains. A Fv fragment may consist of the VL and VH domains of a single arm of an antibody. A dAb fragment (Ward et al. (1989) Nature 341:544-546; incorporated herein by reference) may consist of a VH domain. An isolated complementarity determining region (CDR) having sufficient framework to bind may be an antigen binding portion of a variable region.

### Methods of Assessing Platelet Reactivity

The invention provides methods for assessing platelet reactivity, such as platelet reactivity in a sample comprising platelets from a subject. The invention provides methods for assessing platelet sensitivity, such as platelet sensitivity in a sample comprising platelets from a subject.

The term “platelet function” or “platelet reactivity” as used herein refers to the propensity of platelets to perform functions that are central to haemostasis and thrombosis, such as the formation of aggregates, activation of integrin αllbß3, or the secretion of dense and a granules.

These responses can be triggered by multiple stimuli, such as collagen, adenosine 5′-diphosphate (ADP), and thrombin. In an embodiment, platelet reactivity is indicated by platelet sensitivity and platelet capacity as independent metrics. The term “platelet sensitivity” refers to the sensitivity of a platelet or population of platelets to an agonist. The term “capacity” refers to the capacity or magnitude of a platelet or a population of platelets to generate a functional response.

The methods of the invention enable platelet reactivity or sensitivity to be assessed without the need to actually measure platelet function. Rather, the methods utilise biomarkers that can be readily determined, or measured in a sample from a subject, to provide a measure of platelet reactivity or platelet sensitivity. In one aspect, the invention provides a method of assessing platelet reactivity in a sample comprising platelets from a subject, the method comprising:
(i) determining the level of CD36 in the sample;
(ii) determining the level of GPVI in the sample; and/or
(iii) determining the age of the subject from which the platelets were obtained.

In one aspect, the invention provides a method of assessing platelet sensitivity in a sample comprising platelets from a subject, the method comprising:
(i) determining the level of CD36 in the sample;
(ii) determining the level of GPVI in the sample; and/or
(iii) determining the age of the subject from which the platelets were obtained.

The invention also provides a method for assessing or predicting sensitivity to a platelet activating substance such as CRP (collagen related peptide), such as assessing or predicting sensitivity to a platelet activating substance such as CRP in a sample comprising platelets from a subject, the method comprising:
(i) determining the level of CD36 in the sample;
(ii) determining the level of GPVI in the sample; and/or
(iii) determining the age of the subject from which the platelets were obtained.

The term “platelet activating substance” refers to any substance that is capable of platelet activation. In some embodiments, the platelet activating substance is selected from CRP, CRP-XL, tissue factor, collagen, VWF, PAF, ristocetin, arachidonic acid, adenosine 5′-diphosphate (ADP), thrombin, TRAP-6, epinephrine, and U46619. In some embodiments, the platelet activating substance is a platelet agonist as described herein. In some embodiments, the platelet activating substance is CRP. In some embodiments the platelet activating substance is CRP-XL. In some embodiments, the platelet activating substance is ADP. In some embodiments, the platelet activating substance is epinephrine. In some embodiments, the platelet activating substance is thrombin receptor activator peptide 6 (TRAP-6). In some embodiments, the platelet activating substance is the enzyme thrombin. In some embodiments, the platelet activating substance is U46619 ((5Z)-7-{(1R,4S,5S,6R)-6-[(1E,3S)-3-Hydroxyoct-1-en-1-yl]-2-oxabicyclo[2.2.1]heptan-5-yl}hept-5-enoic acid). Where a platelet activating substance is mentioned herein, it is also intended to encompass more than one platelet activating substance. For example, a subject having sensitivity, such as increased, decreased, high, low, or medium sensitivity, to a platelet activating substance may have that sensitivity to one or more platelet activating substances, such as any one or more platelet activating substances as described herein. The platelet activating substance may refer to CRP and one or more other platelet activating substances. The platelet activating substance may refer to CRP-XL and one or more other platelet activating substances. The platelet activating substance may refer to CRP, ADP, TRAP-6 and/or epinephrine.

The term “sensitivity to a platelet activating substance” refers to the ability of a platelet or a population of platelets to respond to a platelet activating substance, for example by fibrinogen binding (a marker of αllbß3 activation) and/or exposure of P-selectin on the platelet surface (a marker of α-granule secretion). Sensitivity to a platelet activating substance can be used as an indicator of platelet reactivity.

Where the present methods refer to a platelet activating substance, they also encompass more than one platelet activating substance. For example, a method for assessing or predicting sensitivity to a platelet activating substance may assess or predict the sensitivity to one or more platelet activating substances. Any method as described herein for assessing or predicting sensitivity to a platelet activating substance may assess or predict the sensitivity to any one or more platelet activating substances as disclosed herein. For example, the method may assess or predict the sensitivity to CRP and one or more other platelet activating substances. The method may assess or predict the sensitivity to CRP-XL and one or more other platelet activating substances. The method may assess or predict the sensitivity to CRP, ADP, TRAP-6 and/or epinephrine.

The term “CRP” refers to a collagen-related peptide, which is a type of synthetic peptide that mimics the triple-helical structure of collagen and can bind and activate glycoprotein VI (GPVI) receptor for platelet activation. CRPs have a glycine residue every third residue in the peptide to allow folding as a triple helix. An example of a CRP contains a repeat of the amino acid sequence (Gly-Pro-Hyp) or (GPO), for example (Gly-Pro-Hyp)_{10 or} (GPO)₁₀. A CRP may have cysteine residues at the N-terminus and C-terminus which allow for cross-linking (Slatter et al., 2012, Peptides, 36(1):86-89). For example, CRP_{CYS} has Gly-Pro-Cys triplets appended to both the N-terminus and C-terminus. The term “CRP-XL” refers to a cross-linked synthetic collagen-related peptide. Cross-linking enhances the ability of a CRP to form quaternary structure. A cross-linked CRP (CRP-XL) is a more potent platelet agonist than non-cross-linked CRP (Smethurst et al., 2012, J Bio Chem, 282(2):1296-1304). CRP-XL can be cross-linked through cysteine residues at its N-terminus and C-terminus. For example, CRP-XL has the sequence (GPC-(GPO)₁₀-GPCG-amide)₃. An example of a variant of CRP-XL is a synthetic triple helical collagen peptide (STH-CP), which has the same primary sequence as CRP-XL ((GPC-(GPO)₁₀-GPCG-amide)₃), and on both the N-terminus and C-terminus is fixed on a scaffold with a binding side for each of the tree peptides (Sang et al., 2019, Thromb Haemost, 119(12): 2005-2013).

The terms “sensitivity to CRP”, “sensitivity to CRP-XL”, “CRP sensitivity” or “CRP-XL sensitivity” are used interchangeably and refer to the ability of a platelet or a population of platelets to respond to CRP or CRP-XL, respectively, for example by fibrinogen binding (a marker of αllbß3 activation) and/or exposure of P-selectin on the platelet surface (a marker of α-granule secretion). Sensitivity to CRP and CRP-XL can be used as an indicator of platelet reactivity.

The term “high sensitivity” in relation to a platelet activating substance, such as CRP or CRP-XL, refers to an increased ability of a platelet or a population of platelets to respond to that substance, for example by fibrinogen binding (a marker of αllbß3 activation) and/or exposure of P-selectin on the platelet surface (a marker of α-granule secretion) compared to a control or reference platelet or population of platelets.

The term “low sensitivity” in relation to a platelet activating substance, such as CRP or CRP-XL, refers to a decreased ability of a platelet or a population of platelets to respond to that substance, for example by fibrinogen binding (a marker of αllbß3 activation) and/or exposure of P-selectin on the platelet surface (a marker of α-granule secretion) compared to a control or reference platelet or population of platelets.

The term “medium sensitivity” in relation to a platelet activating substance, such as CRP or CRP-XL, refers to a platelet or a population of platelets that do not have high or low sensitivity to that substance.

The inventors have found that a set of biomarkers correlate with sensitivity to a platelet activating substance, such as CRP. Accordingly, in one aspect, the invention provides a method of assessing sensitivity to a platelet activating substance, such as CRP, in a sample comprising platelets from a subject, the method comprising:
(i) determining the level of CD36 in the sample;
(ii) determining the level of GPVI in the sample; and/or
(iii) determining the age of the subject from which the platelets were obtained.

The methods of the invention relate to three biomarkers (also referred to herein as markers), namely the level of CD36, the level of GPVI, and the age of the subject. In the methods of the invention including the methods comprising assessing platelet reactivity and/or assessing or predicting sensitivity to a platelet activating substance, any one, two of three of these markers may be assessed. Accordingly, the methods may comprise assessing:
the level of CD36;
the level of GPVI;
the age of the subject;
the level of CD36 and the level of GPVI;
the level of CD36 and the age of the subject;
the level of GPVI and the age of the subject; or
the level of CD36 and the level of GPVI and the age of the subject.

In the methods of the invention, a step may be included of determining the age of the subject. Such determining may involve obtaining data from a patient record. In the methods of the invention the step of determining the age of the subject may be omitted, for example if the age of the subject is known. For example, in some embodiments the methods may be carried out on a sample from a subject of known age.

In some methods of the invention, the level of CD36 and/or the level of GPVI is determined in a sample. The sample may be a sample obtained from the subject of interest, such as the subject to be determined or the subject to be treated in the relevant method.

The term “sample obtained from” refers to a biological sample originating from a subject.

Where the sample is used for the assessment of one or more biomarkers in platelets, the sample will normally comprise platelets. Accordingly, in the methods of the invention the level of CD36 and/or the level of GPVI may be determined in a sample comprising platelets from the subject. In an embodiment, the sample may be a whole blood sample. In an embodiment, the sample may be plasma. In an embodiment, the sample comprises or consists of platelets from the subject. In an embodiment, the sample comprises or consists of a population of platelets from the subject.

The sample may be provided in vitro. The sample may be a sample that has been obtained from a subject, where the sample is provided in vitro for use in the present methods. The sample may be provided directly or immediately from the subject for use in the methods of the invention, or the sample may be stored under suitable conditions known in the art prior to being used in the methods of the invention.

Where the methods involve determining the level of CD36 and/or GPVI in a sample, and determining the age of the subject, the CD36 and/or GPVI are determined in a sample from the subject whose age is determined. Where the methods of the invention relate to determining or assessing information about a subject, such as a subject's platelet sensitivity, platelet reactivity, sensitivity to a platelet activating substance, risk of a disease, disorder or condition associated with increased platelet reactivity, or suitability for treatment with an anti-platelet therapy, the sample used to assess CD36 and/or GPVI in the methods is a sample from that subject. Where the methods of the invention relate to treatment of a subject, such as treatment of a subject with an anti-platelet therapy, the sample used to assess CD36 and/or GPVI is a sample from that subject.

In some embodiments, the subject has not received anti-platelet therapy. Accordingly, the sample may be from a subject that has not received anti-platelet therapy. For example, the sample may be obtained from the subject before the subject receives any anti-platelet therapy. The subject may have never received anti-platelet therapy. The subject may have not received anti-platelet therapy for a period of at least 1 month, 3 months, 6 months or 1 year prior to the method of the invention, or prior to providing the sample that is used in the present invention. The anti-platelet therapy may refer to any anti-platelet therapy as discussed herein, such as a platelet agglutination inhibitor or a platelet aggregation inhibitor. The anti-platelet therapy may be selected from the group consisting of aspirin, dipyridamole, cyclooxygenase inhibitors (for example, masoprocol, indomethacin, nabumetone, celecoxib, tolmetin, rofecoxib, piroxicam, fenoprofen, valdecoxib, and/or diclofenac), ADP receptor (P2Y) antagonists (for example, clopidogrel, prasugrel, ticagrelor, and/or cangrelor), GPVI inhibitors (for example, glenzocimab, revacept, honokiol, losartan, and/or tussilagone), a drug that targets another platelet receptor, such as a GPIIbIIIa inhibitor (for example, tirofiban and/or eptifibatide), and/or a non-steroidal anti-inflammatory drug (NSAID).

In some embodiments, the subject has no previous history of a disease, disorder or condition associated with increased platelet reactivity. For example, the medical history or medical record of the subject may show no prior occurrence or no prior diagnosis of such a disease, disorder or condition. In some embodiments, the subject does not have a disease, disorder or condition associated with increased platelet reactivity at the time the method of the invention is performed, for example at the time the sample used in a method of the invention was obtained from the subject. The subject may receive or may have received testing to confirm that such a disease, disorder or condition is not present at the time of the method of the invention, for example at the time a sample comprising platelets is obtained for use in the present invention. In some embodiments, the subject has not been diagnosed with a disease, disorder or condition that could be treated with anti-platelet therapy, and/or has not previously been treated with anti-platelet therapy. In an embodiment, the subject has not been diagnosed with a disease, disorder or condition associated with increased platelet reactivity. In some embodiments, the disease, disorder or condition associated with increased platelet reactivity may be any such disease, disorder or condition as described herein, such as a cardiovascular disease, a metabolic or endocrine disease, an abnormal blood condition, an abnormal platelet count, a cancer, a response to a bacterial and/or a viral infection, and/or inflammation. For example, in some embodiments, the subject has no previous history of cardiovascular disease. The cardiovascular disease may be a thrombosis, a myocardial infarction, a stroke, a peripheral arterial disease, an aortic disease, and/or a coronary heart disease.

In some embodiments, the subject is at risk of a disease, disorder or condition associated with increased platelet reactivity. For example, the subject may be or may have been identified by testing, prior medical record, or family history, as having a risk, or an increased risk, of a disease, disorder or condition associated with increased platelet reactivity. The disease, disorder or condition associated with increased platelet reactivity may be any such disease, disorder or condition as described herein, such as a cardiovascular disease, a metabolic or endocrine disease, an abnormal blood condition, an abnormal platelet count, a cancer, a response to a bacterial and/or a viral infection, and/or inflammation. For example, in some embodiments, the subject has a risk or an increased risk, of cardiovascular disease. The cardiovascular disease may be a thrombosis, a myocardial infarction, a stroke, a peripheral arterial disease, an aortic disease, and/or a coronary heart disease. In some embodiments, the subject is or will be, on blood thinning medication, undergoing surgery, and/or a blood donor.

The sample used in the methods of the invention preferably comprises platelets. For example, a sample from a subject comprises platelets from that subject. The sample may be provided directly from the subject, such as a whole blood sample. The sample may be processed prior to use in a method for the invention, such as by removing components. Components may be removed selectively, or may be removed based on particular properties such as size. The sample may be a plasma sample. The sample may be treated or processed to increase the concentration of platelets, for example by removing other components from the sample obtained from the subject. The sample may comprise or consist of platelets from the subject, or may comprise or consist of a population of platelets from the subject.

### Methods of Identifying Subjects

The methods of the invention, such as any of the methods of assessing platelet reactivity and/or assessing or predicting platelet sensitivity, such as sensitivity to a platelet activating substance, as described above, can be used to identify, predict or diagnose a subject having particular characteristics.

In some embodiments is provided a method of identifying a subject having high platelet reactivity or sensitivity to a platelet activating substance. The method comprises:
(i) determining the level of CD36 in a sample comprising platelets from the subject;
(ii) determining the level of GPVI in a sample comprising platelets from the subject; and/or
(iii) determining the age of the subject.

For example, the method may comprise assessing platelet reactivity and/or assessing or predicting platelet sensitivity such as sensitivity to a platelet activating substance in accordance with any such method as disclosed herein. Accordingly, the method may comprise any one, two or three of options (i), (ii) and (iii), such as (i)+(ii), (ii)+(iii), (i)+(iii), or (i)+(ii)+(iii). The sample may be any sample as disclosed herein. The subject may be any subject as disclosed herein. In these methods, the presence of one, two or three of the following is indicative of the presence of high platelet reactivity or sensitivity to a platelet activating substance:
(a) a high level of CD36 in the sample;
(b) a high level of GPVI in the sample; and/or
(c) the subject is at least 50 years of age.

Accordingly, the presence of high platelet reactivity or sensitivity to a platelet activating substance may be indicated by the presence of any one, two or three of options (a), (b) and (c), such as (a)+(b), (b)+(c), (a)+(c), or (a)+(b)+(c). A high platelet reactivity or sensitivity to a platelet activating substance may relate to the platelet reactivity or sensitivity to a platelet activating substance when compared to a control, such as the platelet reactivity or sensitivity to a platelet activating substance in a normal subject or group of subjects, such as a subject or group of subjects having a normal level of platelet reactivity, such as subject(s) not having platelet hypersensitivity or hyposensitivity.

In some embodiments is provided a method of identifying a subject having hypersensitive platelets. The method comprises:
(i) determining the level of CD36 in a sample comprising platelets from the subject;
(ii) determining the level of GPVI in a sample comprising platelets from the subject; and/or
(iii) determining the age of the subject;

For example, the method may comprise assessing platelet reactivity and/or assessing or predicting platelet sensitivity such as sensitivity to a platelet activating substance in accordance with any such method as disclosed herein. Accordingly, the method may comprise any one, two or three of options (i), (ii) and (iii), such as (i)+(ii), (ii)+(iii), (i)+(iii), or (i)+(ii)+(iii). The sample may be any sample as disclosed herein. The subject may be any subject as disclosed herein. In these methods, the presence of one, two or three of the following is indicative of the presence of hypersensitive platelets:
(a) a high level of CD36 in the sample;
(b) a high level of GPVI in the sample; and/or
(c) the subject is at least 50 years of age.

Accordingly, a subject having hypersensitive platelets may be indicated by the presence of any one, two or three of options (a), (b) and (c), such as (a)+(b), (b)+(c), (a)+(c), or (a)+(b)+(c).

The term “hypersensitive platelets” refers to a platelet or a population of platelets with an increased ability to respond to a platelet agonist (e.g., increased sensitivity) and/or is capable of generating a functional response of increased magnitude (e.g., increased capacity) compared to a control or reference platelet or population of platelets. Platelet agonists may induce a signaling-cascade resulting in an activation-dependent conformational change in the major platelet membrane receptor αIIßb3. Examples of platelet agonists include adenosine diphosphate (ADP), thromboxane A2 (TxA2), collagen, thrombin, platelet-activating factor, epinephrine, and serotonin. In some embodiments is provided a method of predicting the responsiveness of a subject to a therapeutic treatment with an anti-platelet therapy. The method comprises:
(i) determining the level of CD36 in a sample comprising platelets from the subject;
(ii) determining the level of GPVI in a sample comprising from the subject; and/or
(iii) determining the age of the subject.

For example, the method may comprise assessing platelet reactivity and/or assessing or predicting platelet sensitivity such as sensitivity to a platelet activating substance in accordance with any such method as disclosed herein. Accordingly, the method may comprise any one, two or three of options (i), (ii) and (iii), such as (i)+(ii), (ii)+(iii), (i)+(iii), or (i)+(ii)+(iii). The anti-platelet therapy may be any anti-platelet therapy as disclosed herein. The sample may be any sample as disclosed herein. The subject may be any subject as disclosed herein.

The term “responsiveness” as used herein refers to the ability to respond or react to a therapeutic treatment, for example, the ability of a subject, a cell (e.g. a platelet), or a population of cells (e.g. a population of platelets) to respond to or react to a therapeutic treatment.

Responsiveness can include the time taken for a subject, cell or population of cells to respond or react to a therapeutic treatment, the level or magnitude of response the therapeutic treatment, or how well a subject, cell or population of cells responds or reacts to a therapeutic treatment.

In some embodiments is provided a method of identifying a subject suitable for treatment with an anti-platelet therapy. The method comprises:
(i) determining the level of CD36 in a sample comprising platelets from the subject;
(ii) determining the level of GPVI in a sample comprising platelets from the subject; and/or
(iii) determining the age of the subject.

For example, the method may comprise assessing platelet reactivity and/or assessing or predicting platelet sensitivity such as sensitivity to a platelet activating substance in accordance with any such method as disclosed herein. Accordingly, the method may comprise any one, two or three of options (i), (ii) and (iii), such as (i)+(ii), (ii)+(iii), (i)+(iii), or (i)+(ii)+(iii). The sample may be any sample as disclosed herein. The subject may be any subject as disclosed herein. In these methods, the presence of one, two or three of the following is indicative that the subject may be suitable for treatment with an anti-platelet therapy:
(a) a high level of CD36 in the sample;
(b) a high level of GPVI in the sample; and/or
(c) the subject is at least 50 years of age.

Accordingly, a subject suitable for treatment with an anti-platelet therapy may be indicated by the presence of any one, two or three of options (a), (b) and (c), such as (a)+(b), (b)+(c), (a)+(c), or (a)+(b)+(c).

Such methods may also be used to select a suitable anti-platelet therapy for the subject, or to select a suitable other therapeutic method for that subject. For example, if a subject is identified as having increased platelet sensitivity or hypersensitive platelets, the subsequent treatment of that patient may be selected to avoid, or to reduce the dose of, a platelet agonist, as any platelet agonist may cause a functional response of increased magnitude in the subject. If a subject is identified as having increased platelet reactivity then the subsequent treatment of that patient may be selected to include, or to increase the dose of, a platelet antagonist or other anti-platelet therapy in order to reduce any unwanted effects or consequences of that increased platelet reactivity.

In some embodiments is provided a method of identifying a subject at reduced risk of bleeding. The method comprises:
(i) determining the level of CD36 in a sample comprising platelets from the subject;
(ii) determining the level of GPVI in a sample comprising platelets from the subject; and/or
(iii) determining the age of the subject.

For example, the method may comprise assessing platelet reactivity and/or assessing or predicting platelet sensitivity such as sensitivity to a platelet activating substance in accordance with any such method as disclosed herein. Accordingly, the method may comprise any one, two or three of options (i), (ii) and (iii), such as (i)+(ii), (ii)+(iii), (i)+(iii), or (i)+(ii)+(iii). The sample may be any sample as disclosed herein. The subject may be any subject as disclosed herein. In these methods, the presence of one, two or three of the following is indicative of a reduced risk of bleeding in the subject:
(a) a high level of CD36 in the sample;
(b) a high level of GPVI in the sample; and/or
(c) the subject is at least 50 years of age.

Accordingly, a reduced risk of bleeding may be indicated by the presence of any one, two or three of options (a), (b) and (c), such as (a)+(b), (b)+(c), (a)+(c), or (a)+(b)+(c).

A reduced risk of bleeding as described herein may be a reduced risk of bleeding in response to treatment with an anti-platelet therapy. For example, some anti-platelet therapies can result in a side-effect of unwanted bleeding in some subjects, which can be difficult to predict but potentially dangerous. Such a reaction or side-effect may be less likely to occur in a subject with increased platelet sensitivity, such as a subject identified by a method as described herein. A reduced risk of bleeding as described herein may be a reduced risk of bleeding in any circumstances where bleeding might occur, such as bleeding as the result of an injury, surgery, or underlying medical condition, such as in a subject receiving blood thinning medication, undergoing surgery, or in a blood donor. A reduced risk of bleeding may be a reduced risk of unwanted bleeding or excessive bleeding. A reduced risk of bleeding may be a reduction in the likelihood that bleeding, such as unwanted bleeding will occur, it may be a reduction in the amount of bleeding, or it may be a reduction in the length of time for which bleeding occurs. A reduced risk of bleeding may relate to the risk of bleeding when compared to a control, such as the risk of bleeding in a normal subject or group of subjects, such as a subject or group of subjects having a normal level of platelet reactivity, such as subject(s) not having platelet hypersensitivity or hyposensitivity. The present methods allow for the identification of subjects where such bleeding, particularly unwanted bleeding or excessive bleeding, is less likely to occur due to increased sensitivity or reactivity of the platelets in the subject. Thus, a subject identified by a method as described herein as having hypersensitive platelets is more likely to have a clotting reaction in response to a bleeding stimulus. This information can be used to select a suitable therapy for use in the subject. For example, a subject identified by a method as described herein as having reduced risk of bleeding can be treated with an anti-platelet therapy, or with an increased dose of anti-platelet therapy. A subject identified by a method as described herein as having a reduced risk of bleeding may be less likely to require antibleeding therapy or prophylaxis.

In some embodiments is provided method of identifying a subject with increased risk of a disease, disorder or condition associated with increased platelet reactivity. The disease, disorder, or condition may be any disease, disorder or condition associated with increased platelet reactivity as disclosed herein. The method comprises:
(i) determining the level of CD36 in a sample comprising platelets from the subject;
(ii) determining the level of GPVI in a sample comprising platelets from the subject; and/or
(iii) determining the age of the subject.

For example, the method may comprise assessing platelet reactivity and/or assessing or predicting platelet sensitivity such as sensitivity to a platelet activating substance in accordance with any such method as disclosed herein. Accordingly, the method may comprise any one, two or three of options (i), (ii) and (iii), such as (i)+(ii), (ii)+(iii), (i)+(iii), or (i)+(ii)+(iii). The sample may be any sample as disclosed herein. The subject may be any subject as disclosed herein. In these methods, the presence of one, two or three of the following is indicative that the subject has increased risk of a disease, disorder or condition associated with increased platelet reactivity:
(a) a high level of CD36 in the sample;
(b) a high level of GPVI in the sample; and/or
(c) the subject is at least 50 years of age.

Accordingly, a subject having increased risk of a disease, disorder or condition associated with increased platelet reactivity may be indicated by the presence of any one, two or three of options (a), (b) and (c), such as (a)+(b), (b)+(c), (a)+(c), or (a)+(b)+(c). An increased risk of a disease, disorder or condition associated with increased platelet reactivity may relate to the risk of a disease, disorder or condition associated with increased platelet reactivity when compared to a control, such as the risk in a normal subject or group of subjects, such as a subject or group of subjects having a normal level of platelet reactivity, such as subject(s) not having platelet hypersensitivity or hyposensitivity.

In some embodiments, the disease, disorder or condition associated with increased platelet reactivity is thrombosis, or a disease, disorder or condition that comprises thrombosis. Thus, the methods described herein can be used to identify a subject with increased risk of thrombosis. An increased risk of thrombosis may relate to the risk of thrombosis when compared to a control, such as the risk of thrombosis in a normal subject or group of subjects, such as a subject or group of subjects having a normal level of platelet reactivity, such as subject(s) not having platelet hypersensitivity or hyposensitivity. The present methods allow for the identification of subjects where thrombosis is more likely to occur due to increased sensitivity or reactivity of the platelets in the subject. Thus, a subject identified by a method as described herein as having hypersensitive platelets is more likely to have an excessive clotting reaction such as thrombosis. This information can be used to select a suitable therapy for use in the subject. For example, a subject identified by a method described herein as having increased risk of thrombosis can be selected for treatment with a therapy intended to reduce platelet reactivity such as an anti-platelet therapy, or with an increased dose of anti-platelet therapy. A subject identified by a method as described herein as having an increased risk of thrombosis may be selected for treatment with an anti-thrombotic therapy such as an anti-platelet therapy or an anticoagulant. For example, a subject identified by a method as described herein as having an increased risk of thrombosis may be treated prophylactically or therapeutically to prevent or reduce thrombosis with an anti-platelet therapy or an anticoagulant therapy. A subject identified by a method as described herein as having an increased risk of thrombosis, and that shows signs or symptoms of thrombosis, may be treated with an anti-platelet therapy or an anticoagulant therapy.

In some embodiments is provided a method of identifying a subject having low platelet reactivity or sensitivity to a platelet activating substance. The method comprises:
(i) determining the level of CD36 in a sample comprising platelets from the subject;
(ii) determining the level of GPVI in a sample comprising platelets from the subject; and/or
(iii) determining the age of the subject.

For example, the method may comprise assessing platelet reactivity and/or assessing or predicting platelet sensitivity such as sensitivity to a platelet activating substance in accordance with any such method as disclosed herein. Accordingly, the method may comprise any one, two or three of options (i), (ii) and (iii), such as (i)+(ii), (ii)+(iii), (i)+(iii), or (i)+(ii)+(iii). The sample may be any sample as disclosed herein. The subject may be any subject as disclosed herein. In these methods, the presence of one, two or three of the following is indicative of the presence of low platelet reactivity or sensitivity to a platelet activating substance:
(a) a low level of CD36 in the sample;
(b) a low level of GPVI in the sample; and/or
(c) the subject is less than 50 years of age.

Accordingly, the presence of low platelet reactivity or sensitivity to a platelet activating substance may be indicated by the presence of any one, two or three of options (a), (b) and (c), such as (a)+(b), (b)+(c), (a)+(c), or (a)+(b)+(c). A low platelet reactivity may relate to the platelet reactivity when compared to a control, such as the platelet reactivity in a normal subject or group of subjects, such as a subject or group of subjects not having platelet hypersensitivity or hyposensitivity.

In some embodiments is provided a method of identifying a subject having hyposensitive platelets. The method comprises:
(i) determining the level of CD36 in a sample comprising platelets from the subject;
(ii) determining the level of GPVI in a sample comprising platelets from the subject; and/or
(iii) determining the age of the subject;

For example, the method may comprise assessing platelet reactivity and/or assessing or predicting platelet sensitivity such as sensitivity to a platelet activating substance in accordance with any such method as disclosed herein. Accordingly, the method may comprise any one, two or three of options (i), (ii) and (iii), such as (i)+(ii), (ii)+(iii), (i)+(iii), or (i)+(ii)+(iii). The sample may be any sample as disclosed herein. The subject may be any subject as disclosed herein. In these methods, the presence of one, two or three of the following is indicative of the presence of hyposensitive platelets:
(a) a low level of CD36 in the sample;
(b) a low level of GPVI in the sample; and/or
(c) the subject is less than 50 years of age.

Accordingly, a subject having hyposensitive platelets may be indicated by the presence of any one, two or three of options (a), (b) and (c), such as (a)+(b), (b)+(c), (a)+(c), or (a)+(b)+(c).

The term “hyposensitive platelets” refers to a platelet or a population of platelets with a decreased ability to respond to a platelet agonist (e.g., decreased sensitivity) and/or is capable of generating a functional response of decreased magnitude (e.g., decreased capacity) compared to a control or reference platelet or population of platelets. A subject having hyposensitive platelets may relate to the sensitivity of platelets when compared to a control, such as a reduced platelet sensitivity when compared to a normal subject or group of subjects, such as a subject or group of subjects having a normal level of platelet reactivity, such as subject(s) not having platelet hypersensitivity or hyposensitivity.

In some embodiments is provided a method of identifying a subject not suitable for treatment with an anti-platelet therapy. The method comprises:
(i) determining the level of CD36 in a sample comprising platelets from the subject;
(ii) determining the level of GPVI in a sample comprising platelets from the subject; and/or
(iii) determining the age of the subject.

For example, the method may comprise assessing platelet reactivity and/or assessing or predicting platelet sensitivity such as sensitivity to a platelet activating substance in accordance with any such method as disclosed herein. Accordingly, the method may comprise any one, two or three of options (i), (ii) and (iii), such as (i)+(ii), (ii)+(iii), (i)+(iii), or (i)+(ii)+(iii). The sample may be any sample as disclosed herein. The subject may be any subject as disclosed herein. In these methods, the presence of one, two or three of the following is indicative that the subject may not be suitable for treatment with an anti-platelet therapy:
(a) a low level of CD36 in the sample;
(b) a low level of GPVI in the sample; and/or
(c) the subject is less than 50 years of age.

Accordingly, a subject not suitable for treatment with an anti-platelet therapy may be indicated by the presence of any one, two or three of options (a), (b) and (c), such as (a)+(b), (b)+(c), (a)+(c), or (a)+(b)+(c).

Such methods may also be used to select a suitable anti-platelet therapy for the subject, or to select a suitable other therapeutic method for that subject. For example, if a subject is identified as having decreased platelet sensitivity or hyposensitive platelets, the subsequent treatment of that patient may be selected to include, or to increase the dose of, a platelet agonist, as any platelet agonist may cause a functional response of reduced magnitude in the subject.

In some embodiments is provided method of identifying a subject with decreased risk of a disease, disorder or condition associated with increased platelet reactivity or sensitivity. The disease, disorder, or condition may be any disease, disorder or condition associated with increased platelet reactivity as disclosed herein. The method comprises:
(i) determining the level of CD36 in a sample comprising platelets from the subject;
(ii) determining the level of GPVI in a sample comprising platelets from the subject; and/or
(iii) determining the age of the subject.

For example, the method may comprise assessing platelet reactivity and/or assessing or predicting platelet sensitivity such as sensitivity to a platelet activating substance in accordance with any such method as disclosed herein. Accordingly, the method may comprise any one, two or three of options (i), (ii) and (iii), such as (i)+(ii), (ii)+(iii), (i)+(iii), or (i)+(ii)+(iii). The sample may be any sample as disclosed herein. The subject may be any subject as disclosed herein. In these methods, the presence of one, two or three of the following is indicative that the subject has a decreased risk of a disease, disorder or condition associated with increased platelet reactivity:
(a) a low level of CD36 in the sample;
(b) a low level of GPVI in the sample; and/or
(c) the subject is less than 50 years of age.

Accordingly, a subject having decreased risk of a disease, disorder or condition associated with increased platelet reactivity may be indicated by the presence of any one, two or three of options (a), (b) and (c), such as (a)+(b), (b)+(c), (a)+(c), or (a)+(b)+(c). A decreased risk of a disease, disorder or condition associated with increased platelet reactivity may relate to the risk of a disease, disorder or condition associated with increased platelet reactivity when compared to a control, such as the risk in a normal subject or group of subjects, such as a subject or group of subjects having a normal level of platelet reactivity, such as subject(s) not having platelet hypersensitivity or hyposensitivity.

In some embodiments, the disease, disorder or condition associated with increased platelet reactivity is thrombosis, or a disease, disorder or condition that comprises thrombosis. Thus, the methods described herein can be used to identify a subject with decreased risk of thrombosis. A decreased or reduced risk of thrombosis may relate to the risk of thrombosis when compared to a control, such as the risk of thrombosis in a normal subject or group of subjects, such as a subject or group of subjects having a normal level of platelet reactivity, such as subject(s) not having platelet hypersensitivity or hyposensitivity. The present methods allow for the identification of subjects where thrombosis is less likely to occur due to decreased sensitivity or reactivity of the platelets in the subject. Thus, a subject identified by a method as described herein as having hyposensitive platelets is less likely to have a clotting reaction such as thrombosis. This information can be used to select a suitable therapy for use in the subject. For example, a subject identified by a method described herein as having reduced risk of thrombosis can be selected for treatment with a therapy having an increased risk of thrombosis as a side effect. A subject identified by a method as described herein as having an increased risk of thrombosis may be less likely to require treatment with an anti-thrombotic therapy such as an anti-platelet therapy or an anticoagulant.

In some embodiments is provided a method of identifying a subject at increased risk of bleeding. The method comprises:
(i) determining the level of CD36 in a sample comprising platelets from the subject;
(ii) determining the level of GPVI in a sample comprising platelets from the subject; and/or
(iii) determining the age of the subject.

For example, the method may comprise assessing platelet reactivity and/or assessing or predicting platelet sensitivity such as sensitivity to a platelet activating substance in accordance with any such method as disclosed herein. Accordingly, the method may comprise any one, two or three of options (i), (ii) and (iii), such as (i)+(ii), (ii)+(iii), (i)+(iii), or (i)+(ii)+(iii). The sample may be any sample as disclosed herein. The subject may be any subject as disclosed herein. In these methods, the presence of one, two or three of the following is indicative of an increased risk of bleeding:
(a) a low level of CD36 in the sample;
(b) a low level of GPVI in the sample; and/or
(c) the subject is less than 50 years of age.

Accordingly, the presence of an increased risk of bleeding may be indicated by the presence of any one, two or three of options (a), (b) and (c), such as (a)+(b), (b)+(c), (a)+(c), or (a)+(b)+(c).

An increased risk of bleeding as described herein may be an increased risk of bleeding in response to treatment with an anti-platelet therapy. For example, some anti-platelet therapies can result in a side-effect of unwanted bleeding in some subjects, which can be difficult to predict but potentially dangerous. An increased risk of bleeding as described herein may be an increased risk of bleeding in any circumstances where bleeding might occur, such as bleeding as the result of an injury, surgery, or underlying medical condition. An increased risk of bleeding may be an increased risk of unwanted bleeding or excessive bleeding. An increased risk of bleeding may be an increase in the likelihood that bleeding, such as unwanted bleeding will occur, it may be an increase in the amount of bleeding, or the length of time for which bleeding occurs. An increased risk of bleeding may relate to the risk of bleeding when compared to a control, such as the risk of bleeding in a normal subject or group of subjects, such as a subject or group of subjects having a normal level of platelet reactivity, such as subject(s) not having platelet hypersensitivity or hyposensitivity. The present methods allow for the identification of subjects where such bleeding, particularly unwanted bleeding or excessive bleeding, is more likely to occur due to decreased sensitivity or reactivity of the platelets in the subject. Thus, a subject identified by a method as described herein as having hyposensitive platelets is less likely to have a clotting reaction in response to a bleeding stimulus.

This information can be used to select a suitable therapy for use in the subject. For example, a subject identified by a method as described herein as having increased risk of bleeding can be treated with a lower dose of anti-platelet therapy. A subject identified by a method as described herein as having an increased risk of bleeding may be more likely to require antibleeding therapy or prophylaxis. A subject identified by a method as described herein as having an increased risk of bleeding may be further treated with an antibleeding therapy or prophylaxis, such as an agent that promotes blood clotting.

### Biomarker Levels

The methods of the invention involve assessing one or more biomarkers, such as CD36 and/or GPVI in a sample comprising platelets from a subject, and/or the age of the subject from which the platelets were obtained.

In some methods of the invention as described herein, for example in a method for identifying a subject having hypersensitive platelets, a method of identifying a subject having increased or high platelet reactivity or sensitivity to a platelet activating substance, a method of identifying a subject with increased risk of a disease, disorder or condition associated with increased platelet reactivity, a method of identifying a subject suitable for treatment with an anti-platelet therapy, or a method of identifying a subject at decreased risk of bleeding, the method may determine the presence of:
(a) a high level of CD36 in the sample;
(b) a high level of GPVI in the sample; and/or
(c) an age of at least 50 years of age.

In some embodiments, the method may determine the presence of:
a high level of CD36 in the sample;
a high level of GPVI in the sample;
an age of at least 50 years of age;
a high level of CD36 in the sample and a high level of GPVI in the sample;
a high level of CD36 in the sample and an age of at least 50 years of age;
a high level of GPVI in the sample and an age of at least 50 years of age; or
a high level of CD36 in the sample and a high level of GPVI in the sample and an age of at least 50 years of age.

In some embodiments, the method may determine the presence of all three of:
(a) a high level of CD36 in the sample;
(b) a high level of GPVI in the sample; and
(c) an age of at least 50 years of age.

In some embodiments, the method may determine the presence of:
(a) a high level of CD36 in the sample; and
(b) a high level of GPVI in the sample;

wherein the subject has an age of at least 50 years of age.

The term “a high level” may refer to a high, higher or increased amount of a protein or nucleic acid molecule of the biomarker compared to a control or reference amount of the protein or nucleic acid molecule of the biomarker.

In an embodiment, a high level of CD36 is defined as a platelet or a population of platelets expressing a level of CD36 that is in at least the top 35^{th} percentile compared to a reference population, for example in the 35^{th}, 34^{th}, 33^{rd}, 32^{nd}, 31^{st}, 30^{th}, 25^{th}, 20^{th}, 15^{th} or 10^{th} percentile.

In an embodiment, a high level of CD36 is defined as being at least 15% higher than the level of CD36 in a control or reference sample comprising platelets, for example at least 16%, at least 17%, at least 18%, at least 19%, at least 20%, at least 25%, at least 30%, at least 35%, at least 45%, or at least 50% higher.

In an embodiment, a high level of CD36 is defined as a platelet or a population of platelets expressing at least 8000 copies of CD36 per platelet, for example at least 8100, at least 8200, at least 8300, at least 8400, at least 8500, at least 8600, at least 8700, at least 8800, at least 8900, at least 9000 or at least 10000 copies of CD36 per platelet.

In an embodiment, a high level of GPVI is defined as a platelet or a population of platelets expressing a level of GPVI that is in at least the top 50^{th} percentile compared to a reference population, for example in the 50 49^{th}, 48^{th}, 47^{th}, 46^{th}, 45^{th}, 40^{th}, 35^{th}, 30^{th}, 25^{th}, 20^{th}, 15^{th} or 10^{th} percentile.

In an embodiment, a high level of GPVI is defined as being at least 15% higher than the level of CD36 in a control or reference sample comprising platelets, for example at least 16%, at least 17%, at least 18%, at least 19%, at least 20%, at least 25%, at least 30%, at least 35%, at least 45%, or at least 50% higher.

In an embodiment, a high level of GPVI is defined as a platelet or a population of platelets expressing at least 3000 copies of GPVI per platelet, for example at least 3100, at least 3200, at least 3300, at least 3400, at least 3500, at least 3600, at least 3700, at least 3800, at least 3900, at least 4000, or at least 5000 copies of GPVI per platelet.

The subject of at least 50 years of age may be at least 51, at least 52, at least 53, at least 54, at least 55, at least 56, at least 57, at least 58, at least 59, at least 60, at least 65, at least 70, at least 75, at least 80, at least 85, or at least 90 years of age. For example, the subject may be at least 50 years or at least 55 years of age. The subject may be 50, 51, 52, 53, 54, 55,56, 57, 58, 59, or 60 years of age. The subject may be 50 to 60, 50 to 70, 50 to 80, 50 to 90, 50 to 100, 55 to 60, 55 to 70, 55 to 80, 55 to 90, 55 to 100, 60 to 70, 60 to 80, 60 to 90, 60 to 100, 70 to 80, 80 to 90, or 90 to 100 years of age.

In an embodiment, a high level of CD36, a high level of GPVI, and/or a subject being at least 50 years of age, is indicative of (a) a subject having hypersensitive platelets; (b) a subject having increased or high platelet reactivity or sensitivity to a platelet activating substance such as CRP or CRP-XL; (c) a subject having increased risk of a disease, disorder of condition associated with increased platelet reactivity; (d) a subject being suitable for treatment with an anti-platelet therapy; (e) a subject being in need of an anti-platelet therapy; and/or (f) a subject at decreased risk of bleeding.

In an embodiment, a high level of CD36 is indicative of: (a) a subject having hypersensitive platelets; (b) a subject having increased or high platelet reactivity or sensitivity to a platelet activating substance such as CRP or CRP-XL; (c) a subject having increased risk of a disease, disorder of condition associated with increased platelet reactivity; (d) a subject being suitable for treatment with an anti-platelet therapy; (e) a subject being in need of an anti-platelet therapy; and/or (f) a subject at decreased risk of bleeding.

In an embodiment, a high level of GPVI is indicative of: (a) a subject having hypersensitive platelets; (b) a subject having increased or high platelet reactivity or sensitivity to a platelet activating substance such as CRP or CRP-XL; (c) a subject having increased risk of a disease, disorder of condition associated with increased platelet reactivity; (d) a subject being suitable for treatment with an anti-platelet therapy; (e) a subject being in need of an anti-platelet therapy; and/or (f) a subject at decreased risk of bleeding.

In an embodiment, a subject being at least 50 years of age is indicative of: (a) a subject having hypersensitive platelets; (b) a subject having increased or high platelet reactivity or sensitivity to a platelet activating substance such as CRP or CRP-XL; (c) a subject having increased risk of a disease, disorder of condition associated with increased platelet reactivity; (d) a subject being suitable for treatment with an anti-platelet therapy; (e) a subject being in need of an anti-platelet therapy; and/or (f) a subject at decreased risk of bleeding.

In an embodiment, a high level of CD36, and a high level GPVI, is indicative of: (a) a subject having hypersensitive platelets; (b) a subject having increased or high platelet reactivity or sensitivity to a platelet activating substance such as CRP or CRP-XL; (c) a subject having increased risk of a disease, disorder of condition associated with increased platelet reactivity; (d) a subject being suitable for treatment with an anti-platelet therapy; (e) a subject being in need of an anti-platelet therapy; and/or (f) a subject at decreased risk of bleeding.

In an embodiment, a high level of CD36, and a subject being at least 50 years of age, is indicative of: (a) a subject having hypersensitive platelets; (b) a subject having increased or high platelet reactivity or sensitivity to a platelet activating substance such as CRP or CRP-XL; (c) a subject having increased risk of a disease, disorder of condition associated with increased platelet reactivity; (d) a subject being suitable for treatment with an anti-platelet therapy; (e) a subject being in need of an anti-platelet therapy; and/or (f) a subject at decreased risk of bleeding.

In an embodiment, a high level of GPVI, and a subject being at least 50 years of age, is indicative of: (a) a subject having hypersensitive platelets; (b) a subject having increased or high platelet reactivity or sensitivity to a platelet activating substance such as CRP or CRP-XL; (c) a subject having increased risk of a disease, disorder of condition associated with increased platelet reactivity; (d) a subject being suitable for treatment with an anti-platelet therapy; (e) a subject being in need of an anti-platelet therapy; and/or (f) a subject at decreased risk of bleeding.

In an embodiment, a high level of CD36, a high level of GPVI, and a subject being at least 50 years of age, is indicative of: (a) a subject having hypersensitive platelets; (b) a subject having increased or high platelet reactivity or sensitivity to a platelet activating substance such as CRP or CRP-XL; (c) a subject having increased risk of a disease, disorder of condition associated with increased platelet reactivity; (d) a subject being suitable for treatment with an anti-platelet therapy; (e) a subject being in need of an anti-platelet therapy; and/or (f) a subject at decreased risk of bleeding.

In an embodiment, a low level of CD36, a high level of GPVI, and a subject being at least 50 years of age, is indicative of: (a) a subject having hypersensitive platelets; (b) a subject having increased or high platelet reactivity or sensitivity to a platelet activating substance such as CRP or CRP-XL; (c) a subject having increased risk of a disease, disorder of condition associated with increased platelet reactivity; (d) a subject being suitable for treatment with an anti-platelet therapy; (e) a subject being in need of an anti-platelet therapy; and/or (f) a subject at decreased risk of bleeding.

In an embodiment, a high level of CD36, a high level of GPVI, and a subject being less than 50 years of age, is indicative of: (a) a subject having hypersensitive platelets; (b) a subject having increased or high platelet reactivity or sensitivity to a platelet activating substance such as CRP or CRP-XL; (c) a subject having increased risk of a disease, disorder of condition associated with increased platelet reactivity; (d) a subject being suitable for treatment with an anti-platelet therapy; (e) a subject being in need of an anti-platelet therapy; and/or (f) a subject at decreased risk of bleeding.

In some methods of the invention as described herein, for example in a method for identifying a subject having hyposensitive platelets, a method of identifying a subject having decreased or low platelet reactivity or sensitivity to a platelet activating substance, a method of identifying a subject with decreased risk of a disease, disorder or condition associated with increased platelet reactivity, a method of identifying a subject not suitable for treatment with an anti-platelet therapy, or a method of identifying a subject at increased risk of bleeding, the method may determine the presence of:
(a) a low level of CD36 in the sample;
(b) a low level of GPVI in the sample;
(c) an age of less than 50 years.

In some embodiments, the method may determine the presence of:
a low level of CD36 in the sample;
a low level of GPVI in the sample;
an age of less than 50 years of age;
a low level of CD36 in the sample and a low level of GPVI in the sample;
a low level of CD36 in the sample and an age of less than 50 years of age;
a low level of GPVI in the sample and an age of less than 50 years of age; or
a low level of CD36 in the sample and a low level of GPVI in the sample and an age of less than 50 years of age.

In some embodiments, the method may determine the presence of all three of:
(a) a low level of CD36 in the sample;
(b) a low level of GPVI in the sample; and
(c) an age of less than 50 years of age.

The method may determine the presence of:
(a) a low level of CD36 in the sample; and
(b) a low level of GPVI in the sample;
wherein the subject has an age of less than 50 years of age.

The term “a low level” refers to a low, lower or decreased number or amount of a protein or nucleic acid molecule of a biomarker compared to a control or reference number or amount of the protein or nucleic acid molecule of the biomarker.

In an embodiment, a low level of CD36 is defined as a platelet or a population of platelets expressing a level of CD36 that is in at least the bottom 65^{th} percentile compared to a reference population, for example in the 65^{th}, 66^{th}, 67 68^{th}, 69^{th}, 70^{th}, 75^{th} or 80^{th} percentile.

In an embodiment, a low level of CD36 is defined as being at least 15% lower than the level of CD36 in a control or reference sample comprising platelets, for example at least 16%, at least 17%, at least 18%, at least 19%, at least 20%, at least 25%, at least 30%, at least 35%, at least 45%, or at least 50% lower.

In an embodiment, a low level of CD36 is defined as a platelet or a population of platelets expressing fewer than 8000 copies of CD36 per platelet, for example fewer than 7900, fewer than 7800, fewer than 7700, fewer than 7600, fewer than 7600, fewer than 6400, fewer than 7300, fewer than 7200, fewer than 7100, fewer than 7000, fewer than 6000, or fewer than 5000 copies of CD36 per platelet.

In an embodiment, a low level of GPVI is defined as a platelet or a population of platelets expressing a level of GPVI that is in at least the bottom 50^{th} percentile compared to a reference population, for example the 50^{th}, 51st, 52nd, 53rd, 54^{th}, 55^{th}, 60^{th}, 65^{th}, or 70^{th} percentile.

In an embodiment, a low level of GPVI is defined as being at least 15% lower than the level of CD36 in a control or reference sample comprising platelets, for example at least 16, at least 17%, at least 18%, at least 19%, at least 20%, at least 25%, at least 30%, at least 35%, at least 45%, or at least 50% lower.

In an embodiment, a low level of GPVI is defined as a platelet or a population of platelets expressing fewer than 3000 copies of GPVI per platelet, for example fewer than 2900, fewer than 2800, fewer than 2700, fewer than 2600, fewer than 2500, fewer than 2400, fewer than 2300, fewer than 2200, fewer than 2100, fewer than 2000, or fewer than 1000 copies of GPVI per platelet. The subject of less than 50 years of age may be less than 50, less than 49, less than 48, less than 47, less than 46, less than 45, less than 44, less than 43, less than 42, less than 41, less than 40, less than 35, less than 35 or less than 30 years of age. In an embodiment, the subject is less than 50 years of age. The subject may be 49, 48, 47, 46, 45, 44, 43, 42, 41, or 40 years of age. The subject may be 0 to 49, 5 to 49, 10 to 49, 15 to 49, 20 to 49, 25 to 49, 30 to 49, 35 to 49, 40 to 49, 45 to 49, 0 to 45, 5 to 45, 10 to 45, 15 to 45, 20 to 45, 25 to 45, 30 to 45, 35 to 45, 0 to 40, 5 to 40, 10 to 40, 15 to 40, 20 to 40, 25 to 40, 30 to 40, 35 to 40 years of age.

In an embodiment, a low level of CD36, a low level of GPVI, and/or a subject being less than 50 years of age, is indicative of: (a) a subject having hyposensitive platelets; (b) a subject having decreased or low platelet reactivity or sensitivity to a platelet activating substance such as CRP or CRP-XL; (c) a subject has decreased risk of a disease, disorder of condition associated with increased platelet reactivity; (d) a subject not being suitable for treatment with an anti-platelet therapy; (e) a subject being in need of an alternative to anti-platelet therapy; and/or (f) a subject being at increased risk of bleeding.

In an embodiment, a low level of CD36 is indicative of: (a) a subject having hyposensitive platelets; (b) a subject having decreased or low platelet reactivity or sensitivity to a platelet activating substance such as CRP or CRP-XL; (c) a subject has decreased risk of a disease, disorder of condition associated with increased platelet reactivity; (d) a subject not being suitable for treatment with an anti-platelet therapy; (e) a subject being in need of an alternative to anti-platelet therapy; and/or (f) a subject being at increased risk of bleeding.

In an embodiment, a low level of GPVI is indicative of: (a) a subject having hyposensitive platelets; (b) a subject having decreased or low platelet reactivity or sensitivity to a platelet activating substance such as CRP or CRP-XL; (c) a subject has decreased risk of a disease, disorder of condition associated with increased platelet reactivity; (d) a subject not being suitable for treatment with an anti-platelet therapy; (e) a subject being in need of an alternative to anti-platelet therapy; and/or (f) a subject being at increased risk of bleeding.

In an embodiment, a subject being less than 50 years of age is indicative of: (a) a subject having hyposensitive platelets; (b) a subject having decreased or low platelet reactivity or sensitivity to a platelet activating substance such as CRP or CRP-XL; (c) a subject has decreased risk of a disease, disorder of condition associated with increased platelet reactivity; (d) a subject not being suitable for treatment with an anti-platelet therapy; (e) a subject being in need of an alternative to anti-platelet therapy; and/or (f) a subject being at increased risk of bleeding.

In an embodiment, a low level of CD36 and a low level of GPVI, is indicative of: (a) a subject having hyposensitive platelets; (b) a subject having decreased or low platelet reactivity or sensitivity to a platelet activating substance such as CRP or CRP-XL; (c) a subject has decreased risk of a disease, disorder of condition associated with increased platelet reactivity; (d) a subject not being suitable for treatment with an anti-platelet therapy; (e) a subject being in need of an alternative to anti-platelet therapy; and/or (f) a subject being at increased risk of bleeding.

In an embodiment, a low level of CD36, and a subject being less than 50 years of age, is indicative of: (a) a subject having hyposensitive platelets; (b) a subject having decreased or low platelet reactivity or sensitivity to a platelet activating substance such as CRP or CRP-XL; (c) a subject has decreased risk of a disease, disorder of condition associated with increased platelet reactivity; (d) a subject not being suitable for treatment with an anti-platelet therapy; (e) a subject being in need of an alternative to anti-platelet therapy; and/or (f) a subject being at increased risk of bleeding.

In an embodiment, a low level of GPVI, and a subject being less than 50 years of age, is indicative of: (a) a subject having hyposensitive platelets; (b) a subject having decreased or low platelet reactivity or sensitivity to a platelet activating substance such as CRP or CRP-XL; (c) a subject has decreased risk of a disease, disorder of condition associated with increased platelet reactivity; (d) a subject not being suitable for treatment with an anti-platelet therapy; (e) a subject being in need of an alternative to anti-platelet therapy; and/or (f) a subject being at increased risk of bleeding.

In an embodiment, a low level of CD36, a low level of GPVI, and a subject being less than 50 years of age, is indicative of: (a) a subject having hyposensitive platelets; (b) a subject having decreased or low platelet reactivity or sensitivity to a platelet activating substance such as CRP or CRP-XL; (c) a subject has decreased risk of a disease, disorder of condition associated with increased platelet reactivity; (d) a subject not being suitable for treatment with an anti-platelet therapy; (e) a subject being in need of an alternative to anti-platelet therapy; and/or (f) a subject being at increased risk of bleeding.

In an embodiment, a low level of CD36, and a high level of GPVI, is indicative of: (a) a subject having hyposensitive platelets; (b) a subject having decreased or low platelet reactivity or sensitivity to a platelet activating substance such as CRP or CRP-XL; (c) a subject has decreased risk of a disease, disorder of condition associated with increased platelet reactivity; (d) a subject not being suitable for treatment with an anti-platelet therapy; (e) a subject being in need of an alternative to anti-platelet therapy; and/or (f) a subject being at increased risk of bleeding.

In an embodiment, a low level of CD36, a low level of GPVI and a subject being at least 50 years of age is indicative of a subject having medium platelet reactivity or sensitivity to a platelet activating substance such as CRP or CRP-XL. In an embodiment, a low level of CD36, a high level of GPVI and a subject being less than 50 years of age is indicative of a subject having medium platelet reactivity or sensitivity to a platelet activating substance such as CRP or CRP-XL. In an embodiment, a high level of CD36, a low level of GPVI and a subject being less than 50 years of age is indicative of a subject having medium platelet reactivity or sensitivity to a platelet activating substance such as CRP or CRP-XL.

A “reference population”, “control” or “reference sample” refers to a control or reference sample or population in which platelet sensitivity or reactivity is normal or in which platelet sensitivity or reactivity is expected to be normal. A determination that a level of a marker of interest is increased relative to that control therefore indicates that the level of the marker is “high” or “increased” or “elevated”. In an embodiment, the reference sample or population is or comprises a platelet, population of platelets or a sample comprising platelets obtained from a donor. In an embodiment, the reference sample or population comprises platelets, populations of platelets or a sample comprising platelets obtained from a population of donors. In an embodiment, the reference sample or population comprises platelets, populations of platelets or samples comprising platelets obtained from multiple donors. Where the reference population, control or reference sample is based on values obtained from multiple donors, the comparison may be with a pooled value such as from a pooled sample. Where the reference population, control or reference sample is based on values obtained from multiple donors, the comparison may be with an average value such as the mean or median value from the donors. The sample or samples used as a reference are preferably the same type of sample used to determine platelet reactivity or sensitivity of the subject in the methods of the invention, such as for example a sample comprising platelets, or a whole blood sample, or a plasma sample. The reference population or reference sample may be from one or more donors known to have a normal level of platelet sensitivity or platelet reactivity, such as no significant increase or decrease in platelet reactivity or sensitivity. The reference population or reference sample may be based on a population of donors of unknown or unassessed platelet sensitivity or platelet reactivity, for example where it is expected that within the population the average level of platelet sensitivity and/or reactivity in the population would be within a normal range.

The donor or donors may or may not have been previously assessed for platelet reactivity or platelet sensitivity by a method of the invention or by any other suitable method. In an embodiment, the donor or population of donors have no previous history of a disease, disorder or condition associated with increased platelet reactivity as described herein, such as selected from the following: a cardiovascular disease, a metabolic or endocrine disease, an abnormal blood condition, an abnormal platelet count, a cancer, a response to a bacterial and/or a viral infection, and/or inflammation. In an embodiment, the donor or population of donors have no previous history of cardiovascular disease. In an embodiment, the donor or population of donors have not previously received an anti-platelet therapy. In an embodiment, the donor or population of donors are not currently taking an anti-platelet therapy. In an embodiment, the donor or population of donors have not been experiencing symptoms of a disease, disorder or condition associated with increased platelet reactivity selected from the following: a cardiovascular disease, a metabolic or endocrine disease, an abnormal blood condition, an abnormal platelet count, a cancer, a response to a bacterial and/or a viral infection, and/or inflammation, when the sample comprising platelets was obtained from the donor.

Where a population of donors is used to provide a reference population, control or reference sample, this may provide a range of values, or set of percentile values, for the reference population, control or reference sample. For example, where the reference population, control or reference sample is based on values from a population of donors, such as a population of donors as described above, the range and spread of values from those donors can be used to assess the relative level of the marker(s) in the sample from the subject. For example, this may be useful to determine whether the level of the marker(s) is increased in the sample from the subject compared to the average (e.g. mean) level in the reference population of donors. This may also be useful to determine whether the level of the marker(s) is increased in the sample from the subject compared to the average (e.g. median) level in the reference population of donors. For example, the level in the sample from the subject may be compared with the levels in the reference population of donors in order to identify the relative position or rank of the sample from the subject within the range of values in the reference population. In some embodiments, the level in the sample from the subject may be allocated to a percentile based on the relative level of the marker in the sample compared with the levels in the reference population. A percentile may be calculated by providing the levels for each donor in the reference population, sorted or ranking them in order of the level of the marker in each subject, and then dividing the ranked values so that one percent of the donors in the population is found in each percentile. The top 1% of donors, having the highest level of the marker, based on the ranking sorted by their level of the marker, form the 1^{st} percentile. The top 5% of donors, based on the ranking sorted by their level of the marker, form the 1^{st}, 2^{nd}, 3^{rd}, 4^{th} and 5^{th} percentiles, which may be described herein as the top 5^{th} percentiles. For example, a ranking that the sample is in the top 5^{th} percentile indicates that up to 5% of the samples in the reference population have a greater level of the marker.

The comparison with a “reference population”, “control” or “reference sample” in the methods of the invention may involve a direct comparison with a suitable reference population, control or reference sample where the relevant marker(s) are determined in the reference population, control or reference sample at the same time or as part of the same method as the sample from the subject. Accordingly, the method of the invention may further comprise (i) determining the level of CD36 in the reference population, control or reference sample; and/or (ii) determining the level of GPVI in the reference population, control or reference sample.

The comparison with a “reference population”, “control” or “reference sample” in the methods of the invention may involve a direct comparison with a suitable reference population, control or reference sample where the relevant marker(s) are determined in the reference population, control or reference sample using the same method or assay used to determine the marker(s) in the sample from the subject. Accordingly, the method of the invention may further comprise (i) determining the level of CD36 in the reference population, control or reference sample by the same assay used to determine the level of CD36 in the sample from the subject; and/or (ii) determining the level of GPVI in the reference population, control or reference sample by the same assay used to determine the level of GPVI in the sample from the subject.

The comparison with a “reference population”, “control” or “reference sample” in the methods of the invention may involve a comparison with known values for the marker(s) in a suitable reference population, control or reference sample such as where the marker(s) have previously been determined in the reference population, control or reference sample, such as where the relevant marker(s) have previously been determined in the reference population, control or reference sample using the same method or assay used to assess the marker(s) in the sample from the subject. For example, where the reference population, control or reference sample is based on a population of donors as described above, a set of standard control values may be obtained for use as a reference population, control or reference sample in the methods of the invention. The set of standard control values may be used as a reference population, control or reference sample in the methods of the invention, e.g. to allow comparison with the level of the marker in the sample from the subject to determine whether the level of the marker in increased or decreased, high or low. The set of standard control values may be used as a reference population, control or reference sample in the methods of the invention, e.g., to allow the ranking of the level in the sample form the subject so that the sample can be allocated to a percentile compared to a reference population, control or reference sample.

The level of CD36 and/or the level of GPVI may be determined by any suitable method.

Such methods may be used to determine the absolute level or the relative level of CD36 and/or GPVI in platelets, the amount of CD36 or GPVI displayed on the platelet membrane, or the levels of soluble CD36 or GPVI in plasma. As used herein, “relative” means that the absolute level is not determined, but a level is determined in comparison to a control or reference sample.

In any of the methods for determining the level of CD36 and/or GPVI described herein, the method may determine the level of CD36 and/or GPVI in a sample as described herein, e.g., a sample of platelets, a whole blood sample or a plasma sample. In addition, in any of the methods for determining the level of CD36 and/or GPVI described herein, the method may determine the level of CD36 and/or GPVI in a control sample, e.g., a sample of platelets, a whole blood sample or a plasma sample, for example a whole blood sample or a plasma sample from a reference donor or population of donors, for example a reference donor or population of donors without a previous history of a disease, disorder or condition associated with increased platelet reactivity.

In some embodiments, the level of CD36 and/or GPVI protein product is determined using an ELISA, a lateral flow test, flow cytometry, an immunological assay, an antibody (or fragment or derivative thereof) assay, a two-site antibody assay, or quantitative mass spectrometry. For example, a two-site immunological assay within an ELISA setting may be used. The term “two-site” means the assay uses two types of antibodies, each of which bind to a different antigen present on the target molecule, e.g. CD36 or GPVI . Typically, one type of antibody is labelled and the other is linked to a solid phase support, allowing precise and reproducible assessment of a target molecule. An example of an ELISA assay for CD36 is described in Allred, C. C., Krennmayr, T., Koutsari, C., Zhou, L., Ali, A. H. and Jensen, M. D., 2011. A novel ELISA for measuring CD36 protein in human adipose tissue. Journal of Lipid Research, 52(2), pp. 408-415.

An example of an ELISA assay for GPVI is described in Al-Tamimi, M., Mu, F. T., Moroi, M., Gardiner, E. E., Berndt, M. C. and Andrews, R. K., 2009. Measuring soluble platelet glycoprotein VI in human plasma by ELISA. Platelets, 20(3), pp. 143-149.

An example of an antibody assay for GPVI is described in Samaha, F. F., Hibbard, C., Sacks, J., Chen, H., Varello, M. A., George, T. and Kahn, M. L., 2004. Measurement of platelet collagen receptor density in human subjects. Arteriosclerosis, Thrombosis, and Vascular Biology, 24(11), pp. e181-e182.

For example, the level of the CD36 and/or GPVI protein product may be determined by measuring the absorbance of UV-light at a wavelength of 280 nm or 205 nm by a sample. Such methods are known in the art, for example as described in Grimsley, G. R. and Pace, C. N., 2003. Spectrophotometric determination of protein concentration. Current protocols in protein science, 33(1), pp.3-1.

For example, the level of the CD36 and/or GPVI protein product may be determined by a colorimetric protein quantification assay, such as a Bradford assay, a bicinchoninic acid assay (BCA), or a Lowry assay, for example as described in Sapan, C. V., Lundblad, R. L. and Price, N. C., 1999. Colorimetric protein assay techniques. Biotechnology and applied Biochemistry, 29(2), pp. 99-108.

For example, the level of the CD36 and/or GPVI protein product may be determined by a NanoOrange fluorescent protein quantification assay, for example as described in Jones, L. J., Haugland, R. P. and Singer, V. L., 2003. Development and characterization of the NanoOrange® protein quantitation assay: a fluorescence-based assay of proteins in solution. Biotechniques, 34(4), pp.850-861.

The presence of the CD36 and/or GPVI protein product may be detected on the basis of its molecular weight, for example by using comparative electrophoretic mobility or mass spectrometry, or using isoelectric focusing.

For example, the level of CD36 and/or GPVI may be determined by measuring the expression of CD36 or GPVI in platelets of the subject. Expression could be measured, for example, by quantitative Western blotting using antibodies specific for the CD36 or GPVI protein. Expression can also be measured, for example, by Northern blotting or reverse-transcription quantitative polymerase chain reaction (RT-qPCR) using probes specific for the CD36 or GPVI mRNA molecule, or by DNA microarrays, for example as described by Ding, Y., Xu, L., Jovanovic, B. D., Helenowski, I. B., Kelly, D. L., Catalona, W. J., Yang, X. J., Pins, M. and Bergan, R. C., 2007. The methodology used to measure differential gene expression affects the outcome. Journal of biomolecular techniques: JBT, 18(5), p.321.

For example, the level of CD36 and/or GPVI protein product may be determined using quantitative mass spectrometry, for example as described by Burkhart, J. M., Vaudel, M., Gambaryan, S., Radau, S., Walter, U., Martens, L., Geiger, J., Sickmann, A., and Zahedi, R. P., 2012. The first comprehensive and quantitative analysis of human plate protein composition allows the comparative analysis of structural and functional pathways. Blood: 120(15), pp. e73-e82.

The expression of CD36 and/or GPVI may also be determined, for example, by using RNA sequencing (RNA-seq), e.g. bulk RNA-seq or single-cell RNA-seq (scRNA-seq) to detect the mRNA transcript of CD36 or GPVI. For example as described in Kukurba, K. R. and Montgomery, S. B., 2015. RNA sequencing and analysis. Cold Spring Harbor Protocols, 2015(11), pp. pdb-top084970.

The expression of CD36 and/or GPVI may also be determined, for example, by using digital PCR (dPCR), to quantify the absolute number of copies of a target DNA or complementary DNA (CDNA) in a sample, for example as described in Pohl, G. and Shih, I. M., 2004. Principle and applications of digital PCR. Expert review of molecular diagnostics, 4(1), pp. 41-47.

The expression of CD36 and/or GPVI may also be determined, for example, by using fluorescence in situ hybridisation (FISH) to detect the CD36 and/or GPVI DNA or mRNA molecules in a sample, for example as described in Young, A. P., Jackson, D. J. and Wyeth, R. C., 2020. A technical review and guide to RNA fluorescence in situ hybridization. PeerJ, 8, p. e8806 and the associated supplementary information.

The level of CD36 and/or GPVI may also be determined using a lateral flow immunochromatographic test, for example, comprising antibodies specific for the CD36 or GPVI protein, for example, as described in Zhou, G., Mao, X. and Juncker, D., 2012. Immunochromatographic assay on thread. Analytical chemistry, 84(18), pp. 7736-7743 and the associated supplementary information.

The level of CD36 and/or GPVI may be determined using flow cytometry, for example, using fluorescent-labeled antibodies specific for the CD36 or GPVI protein, for example as described in Ray, F. A., Friedrich, T. D., Laffin, J. and Lehman, J. M., 1996. Protein Quantitation using flow cytometry. In The Protein Protocols Handbook (pp. 33-38). Humana Press. An example of flow cytometry for measuring CD36 is described in Cserti-Gazdewich, C. M., Dzik, W. H., Dorn, M. E., Quagliaroli, R. O., Xu, S., Ssewanyana, I., Nayyar, R. and Preffer, F. I., 2009. Quantitation of CD36 (platelet glycoprotein IV) expression on platelets and monocytes by flow cytometry: application to the study of Plasmodium falciparum malaria. Cytometry Part B: Clinical Cytometry: The Journal of the International Society for Analytical Cytology, 76(2), pp. 127-134.

The level of CD36 and/or the level of GPVI may be determined indirectly. For example, the activity of CD36 and/or the activity of GPVI may be determined, which may indicate the level of CD36 and/or the level of GPVI respectively. For example, the activity of CD36 and/or the activity of GPVI may be assessed using a flow cytometry-based assay that measures platelet activation in response to a platelet activating substance, and/or a flow cytometry-based assay that measures platelet activation in response to an oxidised lipid agonist of CD36. For example, the activity of CD36 and/or the activity of GPVI may be assessed using a flow cytometry-based assay that measures platelet activation in response to a platelet activating substance and platelet activation in response to an oxidised lipid agonist of CD36.

The methods of the invention are not limited to any particular method for determining the level of CD36 and/or GPVI, and other suitable methods known to the person skilled in the art may also be used to determine the level of CD36 and/or GPVI.

Methods for determining the level of CD36 and/or the level of GPVI may be used in the context of any method of the invention.

### Treatments

The invention further relates to the treatment of subjects as described herein, such as the treatment of subjects whose platelet reactivity or sensitivity to a platelet activating substance has been determined by a method of the invention. The invention relates to the treatment of subjects whose responsiveness to a therapeutic treatment with an anti-platelet therapy has been determined by a method of the present invention. The invention relates to treatment of a subject who has been identified as having hypersensitive platelets by a method of the invention. The invention relates to the treatment of a subject that has been identified as having increased or high platelet reactivity or sensitivity to a platelet activating substance by a method of the invention. The invention relates to the treatment of a subject that has been identified as having increased risk of a disease, disorder or condition associated with increased platelet reactivity by a method of the invention. The invention relates to the treatment of a subject that has been identified as suitable for treatment with an anti-platelet therapy by a method of the invention. The invention relates to the treatment of a subject that has been identified as being at decreased risk of bleeding by a method of the invention. The invention relates to treatment of a subject who has been identified as having hyposensitive platelets by a method of the invention. The invention relates to the treatment of a subject that has been identified as having decreased or low platelet reactivity or sensitivity to a platelet activating substance by a method of the invention. The invention relates to the treatment of a subject that has been identified as having decreased risk of a disease, disorder or condition associated with increased platelet reactivity by a method of the invention. The invention relates to the treatment of a subject that has been identified as not suitable for treatment with an anti-platelet therapy by a method of the invention. The invention relates to the treatment of a subject that has been identified as being at increased risk of bleeding by a method of the invention.

Accordingly, a method of the invention, such as a method of determining responsiveness to a therapeutic treatment with an anti-platelet therapy, a method of identifying a subject having hypersensitive platelets, a method of identifying a subject having increased or high platelet reactivity or sensitivity to a platelet activating substance, a method of identifying a subject having increased risk of a disease, disorder or condition associated with increased platelet reactivity, a method of identifying a subject suitable for treatment with an anti-platelet therapy, or a method of identifying a subject having decreased risk of bleeding, may comprise a further step of administering an anti-platelet therapy to the subject.

A method of the invention, such as a method of determining responsiveness to a therapeutic treatment with an anti-platelet therapy, a method of identifying a subject having hypersensitive platelets, a method of identifying a subject having increased or high platelet reactivity or sensitivity to a platelet activating substance, a method of identifying a subject having increased risk of a disease, disorder or condition associated with increased platelet reactivity, a method of identifying a subject suitable for treatment with an anti-platelet therapy, or a method of identifying a subject having decreased risk of bleeding, may comprise a further step of administering an anti-thrombotic therapy such as an anti-platelet therapy or anticoagulant to the subject.

In one embodiment, the invention provides a method of treating a disease, disorder or condition associated with increased platelet reactivity, the method comprising:
(i) assessing platelet reactivity or sensitivity to a platelet activating substance in a sample comprising platelets from the subject by a method of the invention; and
(ii) administering an anti-platelet therapy to a subject having one, two or three of the following:
   (a) a high level of CD36;
   (b) a high level of GPVI; and/or
   (c) the subject is at least 50 years of age.

In some embodiments, the administration is to a subject having (a) and (b), (b) and (c), (a) and (c), or (a), (b) and (c).

Also provided is a method of treating a subject in need of an anti-platelet therapy, wherein the subject has:
(a) a high level of CD36 in the sample;
(b) a high level of GPVI in the sample; and/or
(c) an age of at least 50 years of age.

In some embodiments, the subject has (a) and (b), (b) and (c), (a) and (c), or (a), (b) and (c). The subject may have all three of:
(a) a high level of CD36 in the sample;
(b) a high level of GPVI in the sample; and
(c) an age of at least 50 years of age.

Where methods of treatment are described herein, the invention also encompasses the equivalent medical uses, such as anti-platelet therapies for use in such methods of treatment. Accordingly, also provided is a method of treating a disease, disorder or condition associated with increased platelet reactivity, the method comprising:
(i) determining the level of CD36 in a sample comprising platelets from the subject;
(ii) determining the level of GPVI in a sample comprising platelets from the subject; and/or
(iii) determining the age of the subject;
   administering the anti-platelet therapy to a subject having one, two or three of the following:
(a) a high level of CD36 in the sample;
(b) a high level of GPVI in the sample; and/or
(c) an age of at least 50 years of age.

In some embodiments, the administration is to a subject having (a) and (b), (b) and (c), (a) and (c), or (a), (b) and (c).

Also provided is an anti-platelet therapy for use in a method of treating a subject in need thereof, the method comprising:
(i) assessing platelet reactivity or sensitivity to a platelet activating substance in a sample comprising platelets from the subject by a method of the invention; and
(ii) administering the anti-platelet therapy to a subject having one, two or three of the following:
(a) a high level of CD36 in the sample;
(b) a high level of GPVI in the sample; and/or
(c) an age of at least 50 years of age.

In some embodiments, the administration is to a subject having (a) and (b), (b) and (c), (a) and (c), or (a), (b) and (c).

Also provided is an anti-platelet therapy for use in a method of treating a subject in need thereof, the method comprising:
(i) determining the level of CD36 in a sample comprising platelets from the subject;
(ii) determining the level of GPVI in a sample comprising platelets from the subject; and/or
(iii) determining the age of the subject;
   administering the anti-platelet therapy to a subject having one, two or three of the following:
(a) a high level of CD36 in the sample;
(b) a high level of GPVI in the sample; and/or
(c) an age of at least 50 years of age.

In some embodiments, the administration is to a subject having (a) and (b), (b) and (c), (a) and (c), or (a), (b) and (c).

Also is provided is an anti-platelet therapy for use in a method of treating a subject in need thereof, wherein the subject has:
(a) a high level of CD36 in the sample;
(b) a high level of GPVI in the sample; and/or
(c) an age of at least 50 years of age.

In some embodiments, the subject has (a) and (b), (b) and (c), (a) and (c), or (a), (b) and (c). The subject may have all three of:
(a) a high level of CD36 in the sample;
(b) a high level of GPVI in the sample; and
(c) an age of at least 50 years of age.
   In any of these treatment methods and medical uses, the method may be a method of treating or preventing a disease, disorder or condition associated with increased platelet reactivity. In any of these treatment methods and medical uses, the anti-platelet therapy may be administered in order to treat or prevent a disease, disorder or condition associated with increased platelet reactivity. The disease, disorder or condition associated with increased platelet reactivity may be any such disease, disorder of condition as disclosed herein. For example, in some embodiments, the anti-platelet therapy may be administered in order to treat or prevent thrombosis. The sample may be any sample as disclosed herein. The subject may be any subject as disclosed herein. The anti-platelet therapy may be as disclosed herein.
   In any of these methods and medical uses, the method may comprise administering the anti-platelet therapy to a subject having any one, two or three of the options (a), (b), and (c), such as (a) and (b), (a) and (c), (b) and (c), or (a), (b) and (c). The method may comprise a step of determining any one, two or three of the options (a), (b), and (c), such as (a) and (b), (a) and (c), (b) and (c), or (a), (b) and (c). The method may determine the presence of any one, two or three of the options (a), (b), and (c), such as (a) and (b), (a) and (c), (b) and (c), or (a), (b) and (c).
   In some embodiments, the subject is at least 50 years of age.
   In some embodiments, the method determines the presence of:
      (a) a high level of CD36 in the sample; and
      (b) a high level of GPVI in the sample;
         wherein the subject is at least 50 years of age.
   In some embodiments, the method determines the presence of all three of:
      (a) a high level of CD36 in the sample;
      (b) a high level of GPVI in the sample; and
      (c) the subject is at least 50 years of age.

Further provided herein is the use of an anti-platelet agent in the manufacture of a medicament for the treatment of a disease, disorder or condition associated with increased platelet reactivity, the treatment comprising:
(i) determining the level of CD36 in a sample comprising platelets from the subject to be treated;
(ii) determining the level of GPVI in a sample comprising platelets from the subject to be treated; and/or
(iii) determining the age of the subject to be treated;
   administering the medicament to a subject having one, two or three of the following:
   (a) a high level of CD36 in the sample;
   (b) a high level of GPVI in the sample; and
   (c) an age of at least 50 years of age.

Also provided herein is the use of an anti-platelet agent in the manufacture of a medicament for the treatment of a disease, disorder or condition associated with increased platelet reactivity, the treatment comprising:
(i) assessing platelet reactivity or sensitivity to a platelet activating substance in a sample comprising platelets from the subject by the method of any one of claims 1-3; and
(ii) administering the medicament to a subject having one, two or three of the following:
(a) a high level of CD36 in the sample;
(b) a high level of GPVI in the sample; and
(c) an age of at least 50 years of age.

Also provided is the use of an anti-platelet agent in the manufacture of a medicament for the treatment of a disease, disorder or condition associated with increased platelet reactivity, wherein the subject has:
(a) a high level of CD36 in the sample;
(b) a high level of GPVI in the sample; and/or
(c) an age of at least 50 years of age.

The subject may have all three of:
(a) a high level of CD36 in the sample;
(b) a high level of GPVI in the sample; and
(c) an age of at least 50 years of age.

The methods and medical uses disclosed herein may also be used to select a suitable anti-platelet therapy for the subject, or to select a suitable other therapeutic method for that subject. For example, if a subject is identified as having increased platelet sensitivity or hypersensitive platelets, the subsequent treatment of that patient may be selected to avoid, or to reduce the dose of, a platelet agonist, as any platelet agonist may cause a functional response of increased magnitude in the subject. If a subject is identified as having increased platelet reactivity then the subsequent treatment of that patient may be selected to include, or to increase the dose of, an anti-platelet agent or other anti-platelet therapy in order to reduce any unwanted effects or consequences of that increased platelet reactivity. Where the methods described herein relate to the administration of an anti-platelet therapy, the anti-platelet therapy may be any suitable anti-platelet therapy as described herein. Where the methods described herein relate to the administration of an anti-platelet therapy, the anti-platelet therapy may be replaced by or combined with any other anti-thrombotic therapy. For example, if a subject is identified as having increased platelet sensitivity or hypersensitive platelets, the treatment of that patient may be selected to include, or to increase the dose of, an anti-thrombotic agent such as an anti-platelet therapy or anticoagulant. The treatment of that patient may be selected to include, or to increase the reliance on or dose of, an anti-platelet agent or other anti-platelet therapy or other antithrombotic. If a subject is identified as having increased platelet sensitivity or hypersensitive platelets, the treatment of that patient may be selected to avoid, or to reduce the reliance on or dose of, an agent that may promote or increase the risk of thrombosis.

Also provided is the use of an alternative to an anti-platelet therapy in a method of treating a subject in need thereof, wherein the subject has:
(a) a low level of CD36 in the sample;
(b) a low level of GPVI in the sample; and/or
(c) an age of less than 50 years of age.

The subject may have all three of:
(a) a low level of CD36 in the sample;
(b) a low level of GPVI in the sample; and
(c) an age of less than 50 years of age.
   The method may comprise administering the alternative anti-platelet therapy to a subject having any one, two or three of the options (a), (b), and (c), such as (a) and (b), (a) and (c), (b) and (c), or (a), (b) and (c). The method may comprise a step of determining any one, two or three of the options (a), (b), and (c), such as (a) and (b), (a) and (c), (b) and (c), or (a), (b) and (c). The method may determine the presence of any one, two or three of the options (a), (b), and (c), such as (a) and (b), (a) and (c), (b) and (c), or (a), (b) and (c).

Such methods may be used to select a suitable anti-platelet treatment for the subject, or to select a suitable other therapeutic method for that subject. For example, if a subject is identified as having decreased platelet sensitivity or hyposensitive platelets, the subsequent treatment of that patient may be selected to include, or to increase the dose of, a platelet agonist, as any platelet agonist may cause a functional response of reduced magnitude in the subject. The subsequent treatment of that patient may be selected to exclude, or to reduce the reliance on or dose of, an anti-platelet agent or other anti-platelet therapy, as an anti-platelet therapy is less likely to be beneficial in a subject having decreased platelet sensitivity or reactivity. If a subject is identified as having decreased platelet sensitivity or hyposensitive platelets, the treatment of that patient may be selected to avoid, or to reduce the reliance on or the dose of, an anti-thrombotic agent such as an anti-platelet therapy or anticoagulant. If a subject is identified as having increased platelet sensitivity or hypersensitive platelets, the subject may be more susceptible to bleeding, such as unwanted or excessive bleeding. Accordingly, if a subject is identified as having increased platelet sensitivity or hypersensitive platelets, the treatment of that patient may be selected to avoid, or to reduce the reliance on or dose of, an agent that may promote or increase the risk of bleeding. if a subject is identified as having increased platelet sensitivity or hypersensitive platelets, the treatment of that patient may be selected to include, or to increase the reliance on or dose of, an agent or other therapy that may treat or prevent bleeding.

The term “effective amount” or “therapeutically effective amount” are used interchangeably herein, and refer to an amount of a compound, formulation, material, or composition, as described herein effective to achieve a particular biological result.

As used herein, the terms “treat”, “treatment” and “treating” refer to the reduction or amelioration of the progression, severity and/or duration of a disease, disorder or condition, or the amelioration of one or more symptoms (preferably, one or more discernible symptoms) of a disease, disorder or condition. In specific embodiments, the terms “treat”, “treatment” and “treating” refer to the amelioration of at least one measurable physical parameter of a disease, disorder or condition associated with increased platelet reactivity, for example selected from the following: a cardiovascular disease, a metabolic or endocrine disease, an abnormal blood condition, an abnormal platelet count, a cancer, a response to a bacterial and/or a viral infection, and/or inflammation, such as growth of a thrombus, not necessarily discernible by the patient. In other embodiments the terms “treat”, “treatment” and “treating” refer to the inhibition of the progression of a disease, disorder or condition associated with increased platelet reactivity, either physically by, for example, stabilization of a discernible symptom, physiologically by, for example, stabilization of a physical parameter, or both. In other embodiments the terms “treat”, “treatment” and “treating” refer to the reduction or stabilization of thrombus size. In some embodiments, the terms “treat”, “treatment” and “treating” encompass prophylactic treatment to prevent the onset, progression, worsening, or relapse of a disease, disorder or condition associated with increased platelet reactivity, or symptoms of a disease, disorder or condition associated with increased platelet reactivity.

A treatment as described herein may be therapeutic or prophylactic. The term “therapeutic” as used herein means a treatment. A therapeutic effect is obtained by reduction, suppression, remission, or eradication of a disease state. The term “prophylaxis” or “prophylactic” as used herein means the prevention of or protective treatment for a disease or disease state.

The term “anti-platelet therapy” refers to a therapy intended to decrease platelet aggregation and/or inhibit thrombus formation. In an embodiment, the anti-platelet therapy may be a platelet agglutination inhibitor or a platelet aggregation inhibitor. In an embodiment, the anti-platelet therapy may be selected from the group consisting of aspirin, dipyridamole, cyclooxygenase inhibitors (for example, masoprocol, indomethacin, nabumetone, celecoxib, tolmetin, rofecoxib, piroxicam, fenoprofen, valdecoxib, and/or diclofenac), ADP receptor (P2Y) antagonists (for example, clopidogrel, prasugrel, ticagrelor, and/or cangrelor), GPVI inhibitors (for example, glenzocimab, revacept, honokiol, losartan, and/or tussilagone), a drug that targets another platelet receptor, such as a GPIIbIIIa inhibitor (for example, tirofiban and/or eptifibatide), and/or a non-steroidal anti-inflammatory drug (NSAID). Other anti-platelet therapies may be known to the skilled person. In an embodiment of methods disclosed herein, administration of the anti-platelet therapy does not occurs prior to the method of the invention. In an embodiment of methods disclosed herein, administration of the anti-platelet therapy occurs after the method of the invention. The timing of administration is not fixed and in other embodiments may occur before or during the method of the invention.

The term “a subject in need of an anti-platelet therapy” refers to a subject (e.g., a patient) who has, or is at risk of, a disease, disorder or condition that could be treated with anti-platelet therapy, or a subject who is, or will be, on blood thinning medication, undergoing surgery, and/or a blood donor, and may benefit from anti-platelet therapy. Particular but non-limiting examples of surgery a subject may be undergoing or will be undergoing include: major surgical procedures known for a high risk of significant blood loss, cardio-thoracic surgery, orthopedic surgery, organ transplantation (e.g., liver, heart, lung, and/or kidney), surgery for trauma, brain surgery (e.g. including invasive imaging) and/or vessel surgery. A subject in need of an anti-platelet therapy may have elevated levels of platelet reactivity that may result in harmful effects. Treatment of the subject with anti-platelet therapy may reduce the platelet reactivity in the subject. Treatment of the subject with anti-platelet therapy may reduce the onset or development of harmful effects caused by the increased platelet reactivity in the subject. Treatment of the subject with anti-platelet therapy may reduce the response of hypersensitive platelets in the subject to a stimulus, such as a stimulus that would provoke blood clotting, such as surgery.

As used herein, the term “disease, disorder or condition” refers to a pathological disease, disorder or condition, for example associated with increased platelet reactivity. In an embodiment, the disease, disorder or condition associated with increased platelet reactivity is a cardiovascular disease (CVD), a metabolic or endocrine disease, an abnormal blood condition, an abnormal platelet count, a cancer, a response to a bacterial and/or a viral infection, and/or inflammation. In an embodiment, the cardiovascular disease is a thrombosis, a disease, disorder or condition comprising or associated with thrombosis, a myocardial infarction, a stroke, a peripheral arterial disease, an aortic disease, and/or a coronary heart disease (CHD). In an embodiment, the subject is or will be on blood thinning medication, undergoing surgery, and/or a blood donor.

The term “an abnormal platelet count” as used herein refers to a low platelet count (e.g., <50×10⁹/L. In some embodiments, an abnormal or low platelet count may be caused by reduced generation of platelets by bone marrow (e.g., in cancer patients because of bone marrow infiltration or iatrogenic), increased destruction of platelets (e.g. patients with antibody-mediated thrombocytopenias), increased consumption (e.g., patients with disseminated intravascular coagulation), or hyperslenism.

The term “an abnormal blood condition” as used herein refers to a condition where blood has abnormal characteristics or functioning. In some embodiments, the abnormal blood condition may be an acquired or congenital platelet function defect and/or an inherited bleeding disorder, including, but not limited to, disseminated intravascular coagulation (DIC), prothrombin deficiency, Factor V, VII, or X deficiency, Factor XI deficiency (haemophilia C), haemophilia A, haemophilia B, idiopathic thrombocytopenia purpura (ITP), Von Willebrand disease (types I, II, and III), Glanzmann thrombasthenia, and/or an acquired coagulopathy such as liver disease and/or viral hepatitis.

The term “cancer” refers to a disease characterized by the rapid and uncontrolled growth of aberrant cells. Cancer cells can spread locally or through the bloodstream and lymphatic system to other parts of the body. Examples of various cancers are described herein and include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. In some embodiments cancers treated by the methods described herein include multiple myeloma, Hodgkin's lymphoma or non-Hodgkin's lymphoma. The terms “tumor” and “cancer” are used interchangeably herein, for example, both terms encompass solid and liquid, for example, diffuse or circulating, tumors. As used herein, the term “cancer” or “tumor” includes premalignant, as well as malignant cancers and tumors.

The term “suitable for treatment with an anti-platelet therapy” refers to a subject identified as being likely to respond to treatment with an anti-platelet therapy. In an embodiment, the subject may be identified as being sensitive to a platelet activating substance such as CRP or CRP-XL. In an embodiment, the subject may be identified as being highly sensitive to a platelet activating substance such as CRP or CRP-XL. In an embodiment, the subject may be identified as having a high level of CD36 expression by a population of platelets of the subject. In an embodiment, the subject may be identified as having a high level of GPVI expression by a population of platelets of the subject. In an embodiment, the subject may be identified as being at least 50 years of age. In an embodiment, the subject may be identified as being at least 55 years of age.

The term “not suitable for treatment with an anti-platelet therapy” refers to a subject identified as being unlikely to respond to treatment with an anti-platelet therapy. In an embodiment, the subject may be identified as not being sensitive to a platelet activating substance such as CRP-XL. In an embodiment, the subject may be identified as not being highly sensitive to a platelet activating substance such as CRP-XL. In an embodiment, the subject may be identified as having a low level of CD36 expression by a population of platelets of the subject. In an embodiment, the subject may be identified as having a low level of GPVI expression by a population of platelets of the subject. In an embodiment, the subject may be identified as being under 55 years of age. In an embodiment, the subject may be identified as being under 50 years of age. In an embodiment, the subject not suitable for treatment with an anti-platelet therapy is administered an alternative therapy to an anti-platelet therapy. In an embodiment, the subject not suitable for treatment with an anti-platelet therapy is administered an altered dose of anti-platelet therapy as disclosed herein, e.g., an increased or decreased dose of an anti-platelet therapy as disclosed herein.

### Kits

Also provided herein is a kit which comprises means for determining the level of CD36 and/or GPVI in a platelet or a population of platelets of a subject.

Also provided herein is a kit for determining whether a subject has, or is at risk of, a disease, disorder or condition associated with increased platelet reactivity, which comprises means for determining the level of CD36 and/or GPVI in a platelet or a population of platelets of a subject.

The determining means may include an antibody, or fragment or derivative thereof, which is specific to the protein expression product of CD36 or GPVI. The determining means may include an antibody, or fragment or derivative thereof, which is specific to CD36 and an antibody, or fragment or derivative thereof, which is specific to GPVI.

The kits may be provided with all the reagents necessary for the detection of the CD36 and/or GPVI molecule. For example, the kits may include labels for detection of bound antibodies, or probes, reagents for isolating or obtaining mRNA or cDNA from a biological sample obtained from the subject.

The kits may be provided with instructions for the use thereof as a test to determine whether a subject has, or is at risk of, a disease, disorder or condition associated with increased platelet reactivity.

### EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### Example 1: Identification and Validation Of CD36, GPVI and Age as Biomarkers of Platelet Function

### Methods

### Blood Donation and Cohorts

The study was carried out in accordance with the Declaration of Helsinki. Recruitment of healthy fasted non-smoking donors aged 30 to 65 years and free from drugs known to influence platelet function (cohort 1) was approved by the University of Reading Research Ethics Committee. Recruitment of nonfasted donors aged 18 to 75 years (cohort 2) was approved by the Cambridge East Research Ethics Committee.

The biomarkers were determined using platelet samples from healthy volunteers. The analysis has also been validated using patients with cardiovascular disease with and without diabetes. All samples were taken with written informed consent using processes approved by the University and NHS ethics committees (as required) and for patients, approval was also granted by the Health Research Authority.

Patient samples used to validate the biomarkers were obtained within an ongoing clinical study in collaboration with the University Department of Cardiology at the Royal Berkshire NHS Foundation Trust.

### Platelet Function Assay

The flow cytometry assay measured fluorescein isothiocyanate-conjugated anti-fibrinogen antibody and PE-Cy5-conjugated anti-P-selectin antibody binding following stimulation with ADP (0.03-30 mM), collagen-related peptide (CRP; 0.003-3 mg/mL), epinephrine (0.1-30 mM), thrombin receptor activator peptide 6 (TRAP-6; 0.05-15 mM), and U46619 (0.3-100 mM) using an Accuri C6 (B D, Winnersh, UK) for cohort 1 and a Beckman-Coulter EPICS Profile XL for cohort 2, as described in supplemental Section 2 of Dunster 2021 (Dunster et al. Multiparameter phenotyping of platelet reactivity for stratification of human cohorts, Blood Adv (2021) 5(20): 4017-4030).

### PPAnalysis of Platelet Sensitivity and Capacity

A Platelet Phenomic Analysis (PPAnalysis) assay was carried out described in Dunster 2021. PPAnalysis comprises a flow cytometry-based assay and bespoke software that was developed to make data analysis simple, rapid, and reproducible. The assay simultaneously measures 2 aspects of platelet activation, fibrinogen binding (a marker of αllßb3 activation) and the exposure of P-selectin on the platelet surface (a marker of a-granule secretion). The supplementary information accompanying Dunster 2021 provides step-by-step instructions for setting up and carrying out the PPAnalysis assay and data analysis.

### In Vitro Thrombus Formation Assay

Thrombus formation on immobilized type I collagen was performed using the VenaFlux Platform and Vena 8Fluor+Biochips (Cellix). Channels were coated overnight at 4° C. with collagen, then blocked with 1% BSA/PBS for 1 h and replaced with PBS. Blood was collected into sodium citrate (4% w/v). Platelets in whole blood were incubated with 10 mM DiOC6 (Thermo Fisher Scientific) for 10 min. Perfusion was performed at 37° C. with an arterial shear rate of 1000 s⁻¹ for at least 520 seconds. Thrombus formation was visualised using a 20× magnification lens on a Nikon A1-R confocal microscope. Images were brightness corrected and converted to a binary mask with software provided auto-thresholds. Binary Mask Particles were then analysed to determine thrombus count and size.

### Identifying Biomarkers Using Statistical Analyses

A bidirectional stepwise linear regression was used to identify possible predictors of sensitivity to a platelet activating substance such as CRP-XL out of a range of candidate variables. At each step, variables were added based on p-values, and the AIC was used to set a limit on the total number of variables included in the final model. Age, CD36 and GPVI expression were identified as predictors of sensitivity to a platelet activating substance such as CRP-XL.

### Measuring Biomarkers

To measure the levels, e.g. the number of copies, of CD36 and GPVI expressed by platelets, a two-site immunological assay within an ELISA setting is used. Surface levels of CD36 and GPVI can also be measured by flow cytometry.

### Results

### Sensitivity to Platelet GPVI Stimulation Predicts Thrombus Formation on Collagen

Variance in the growth of platelet thrombi in vitro on type I collagen was measured in a cohort of 27 healthy donors by perfusing citrated whole blood through flow chambers at an arterial shear rate of 1000s⁻¹ (FIG. 1A). Thrombus growth occurred at a range of different rates (FIG. 1Bi) and a large degree of variance (7.4-fold between largest and smallest thrombi) was apparent after 520 seconds (FIG. 1Bii). Platelet reactivity was also investigated in the same cohort of healthy donors by measuring p-selectin exposure in response to TRAP-6, ADP and CRP-XL (FIG. 1C) using PPAnalysis, which provides two outputs of reactivity for each agonist: sensitivity and capacity (FIG. 1D). sensitivity to TRAP-6 was the least variable and sensitivity to CRP-XL was highly variable, with ADP having intermediate variance (FIG. 1Ei). The variance in capacity was similar for all three agonists (FIG. 1Eii). Comparison of each measure of reactivity to thrombus size using Pearson's correlation (FIG. 1F) demonstrated a significant correlation with sensitivity for CRP-XL only (FIG. 1Fvi).

Age and Platelet Surface Expression of GPVI and CD36 Predicts Sensitivity to Platelet Activating substances

Measurement of in vitro thrombus formation is impractical in a clinical setting and, even incorporation of a measure of platelet reactivity such as PPAnalaysis into clinical trials or routine clinical practice represents a major financial and logistical challenge. Identification of biomarkers associated with these functional measures will facilitate clinical utilisation. Biomarkers potentially associated with sensitivity for platelet activating substances were investigated in a large cohort of 461 healthy donors. Measurement of p-selectin exposure in response to TRAP-6, ADP and CRP-XL using PPAnalysis identified the same trends in the variance of sensitivity and capacity (FIG. 2A) identified previously in the smaller cohort (FIG. 1E). Surface expression of a range of platelet surface proteins with a potential to influence platelet reactivity were also measured by flow cytometry and age, gender, body mass index (BMI), platelet count and mean platelet volume (MPV in femtolitres, fL) were also collected (FIG. 2B-C). In a randomly selected training cohort (FIG. 2D) investigating 18 features as potential biomarkers of sensitivity to platelet activating substances, a bidirectional stepwise regression model identified three features, age, CD36 and GPVI expression, as predictors (FIG. 2E). Associations between GPVI expression, CD36 expression and age versus sensitivity to CRP-XL were investigated using Pearson's correlation in the test cohort and found to be significant (FIG. 2Fi-iii). The multiple regression model identified in the training cohort was used to predict the sensitivity to CRP-XL of the test cohort using their biomarker parameters (predicted sensitivity to CRP-XL) and then plotted against the measured sensitivity to CRP-XL (FIG. 2Fiv).

### Correlation Between CD36 Expression of Sensitivity to Platelet Activating Substances Increases With Age

Further investigation of the association between CD36 expression and sensitivity to CRP-XL showed that the association is weaker those aged <55 years (r²=0.19) than those aged ≥55 years (r²=0.36, FIG. 3A). The association between GPVI receptor expression and sensitivity to CRP-XL was similar in those aged <55 s (r²=0.34) and ≥55 (r²=0.38). This finding suggests that CD36 and GPVI expression has similar predictive value for sensitivity to CRP-XL, which is surprising considering that CRP-XL is a GPVI specific ligand. The ability of GPVI and CD36 expression to predict high sensitivity to platelet activating substances such as CRP-XL (top 33^{rd} percentile) was investigated in the two age groups using ROC analysis (FIG. 3C). The area under the curve (AUC) for CD36 (55.0%) was lower than GPVI (66.0%) in the <55 age group but in the ≥55 group CD36 (72.2%) was higher than GPVI (65.8%). CD36 expression may therefore be of high predictive value in patients with ischaemic heart disease, which becomes more prevalent in older age.

### Stratification of Sensitivity to Platelet Activating Substances Using Age, CD36 and GPVI Surface Expression

The inventors designed a stratification algorithm using age and expression of CD36 and GPVI to predict low, medium or high sensitivity to platelet activating substances (FIG. 4A). The first step stratifies based on an age cut-off of 55 years and all those over 55 years are ultimately predicted to have either medium or high sensitivity. If age is ≥55 and CD36 expression is high (top 33^{rd} percentile) then the sensitivity is predicted to be high, if CD36 is low but GPVI is high (top 50^{th} percentile) then sensitivity is also predicted to be high, but if GPVI is also low sensitivity is predicted to be medium. In the <55 both CD36 and GPVI expression must be high for sensitivity to platelet activating substances to be predicted to be high. If either CD36 or GPVI is high, then sensitivity is predicted to be medium. Only if age <55 and both CD36 and GPVI are low is the sensitivity predicted to be low. When stratification algorithm was applied to the cohort, three groups with significantly different sensitivity to platelet activating substances (CRP-XL) were identified, in line with the predication (FIG. 4B).

### Stratification of Sensitivity to Platelet Activating Substances and In Vitro Thrombus Formation in a Cohort of Patients With Ischemic Heart Disease

The inventors proceeded to test the stratification algorithm in a cohort of 38 patients with ischaemic heart disease by measuring CD36 and GPVI expression and performing PPAnalysis. Sensitivity to platelet activating substances (CRP-XL) correlated with significantly with expression of CD36 and GPVI (FIG. 5A&B). However the association between sensitivity to platelet activating substances and age was not significant in this cohort of patients (FIG. 5C). It was hypothesised that aspirin use might be more prevalent in older patients and that this might counter-act the positive association between age and sensitivity to platelet activating substances. The average age of patients taking ASA (65±8) was significantly higher than patients free of ASA (58±13, FIG. 5D)) and the sensitivity to CRP-XL of patients receiving ASA (7.2±0.37 log g/mL) was significantly lower than ASA free patients (7.4±0.49 log g/mL, FIG. 5E) suggesting that aspirin use in older patients counter-acted the expected association between sensitivity to CRP-XL and age. The stratification algorithm predicted only 4 patients to have low sensitivity to CRP-XL due to generally older age of patients (n=8 patients under 55) withing the cohort (FIG. 5F). Differences in mean sensitivity to CRP-XL of the patients predicted to have high or medium sensitivity to CRP-XL were significant, but comparisons with patients predicted to have low sensitivity were not significant, possibly due to low numbers in this group.

The volumes of thrombi formed after perfusing whole blood over type I collagen for 6 minutes at an arterial shear rate of 1000 s⁻¹ were measured to investigate the potential of the stratification algorithm to identify patients prone to forming large thrombi. First, the sensitivity to CRP-XL was compared to thrombus volume to confirm that the relationship identified in FIG. 1Fiii was also present in this cohort of patients and found that the association was significant (FIG. 6A). The inventors next investigated whether the individual components of the stratification algorithm correlated with thrombus volume. There was a significant association of GPVI and CD36 expression with thrombus volume (FIG. 6B&C). In contrast to the lack of significant association between sensitivity to CRP-XL and age on this cohort of patients, there was a significant correlation between thrombus volume and age (FIG. 6F). The hypothesis that the increase in platelet reactivity associated with age is counter-acted by more frequent use of aspirin in older patients did not appear to apply to thrombus volume measurements, which were not significantly different in patients with or without aspirin (FIG. 6G). As the individual components of the stratification algorithm were associated with thrombus volume, the algorithm was applied to these data, finding that patients predicted to have high sensitivity to CRP-XL also had significantly larger thrombi that patients predicated to have medium or low (FIG. 6G&I).

### REFERENCES

Bigalke B, Haap M, Stellos K, Geisler T, Seizer P, Kremmer E, Overkamp D, Gawaz M. Platelet glycoprotein VI (GPVI) for early identification of acute coronary syndrome in patients with chest pain. Thromb Res. 2010;125(5): e184-189.
Dunster J L, Bye A P, Kriek N, Sage T, Mitchell J L, Kempster C, Batista J, Mckinney H, Thomas P, Jones C I, Downes K, Unsworth A J, Gibbins J M. Multiparameter phenotyping of platelet reactivity for stratification of human cohorts. Blood Adv. 2021 Oct. 26;5(20):4017-4030.
Knowles J W, Wang H, Itakura H, Southwick A, Myers R M, Iribarren C, Fortmann S P, Go A S, Quertermous T, Hlatky M A. Association of polymorphisms in platelet and hemostasis system genes with acute myocardial infarction. Am Heart J. 2007;154(6):1052-1058.
Sherva R, Miller M B, Pankow J S, Hunt S C, Boerwinkle E, Mosley T H, Weder A B, Curb J D, Luke A, Morrison A C, Fornage M, Arnett D K. A whole-genome scan for stroke or myocardial infarction in family blood pressure program families. Stroke. 2008;39(4):1115-1120.

### EQUIVALENTS

The disclosures of each and every patent, patent application, and publication cited herein are hereby incorporated herein by reference in their entirety. While this invention has been disclosed with reference to specific aspects, it is apparent that other aspects and variations of this invention may be devised by others skilled in the art without departing from the true spirit and scope of the invention. The appended claims are intended to be construed to include all such aspects and equivalent variations.

1. A method of assessing platelet reactivity or sensitivity to a platelet activating substance in a sample comprising platelets from a subject, the method comprising:
(i) determining the level of CD36 in the sample;
(ii) determining the level of GPVI in the sample; and/or
(iii) determining the age of the subject from which the platelets were obtained.

2. The method of claim 1, wherein the method comprises:
(I) (i) determining the level of CD36 in the sample; and
(ii) determining the level of GPVI in the sample; or
(II) (i) determining the level of CD36 in the sample; and
(ii) determining the age of the subject from which the platelets were obtained; or
(III) (i) determining the level of GPVI in the sample; and
(ii) determining the age of the subject from which the platelets were obtained.

3. The method of claim 1, wherein the method comprises:
(i) determining a level of CD36 in the sample;
(ii) determining the level of GPVI in the sample; and
(iii) determining the age of the subject.

4. A method of predicting the responsiveness of a subject to a therapeutic treatment with an anti-platelet therapy, the method comprising:
(i) determining the level of CD36 in a sample comprising platelets from the subject;
(ii) determining the level of GPVI in a sample comprising platelets from the subject; and/or
(iii) determining the age of the subject.
optionally wherein the method comprises assessing platelet reactivity or sensitivity to a platelet activating substance in a sample comprising platelets from the subject by the method of any one of claims 1 to 3.

5. A method of identifying a subject having hypersensitive platelets, the method comprising assessing platelet reactivity or sensitivity to a platelet activating substance in a sample comprising platelets from the subject by the method of any one of claims 1-3;
wherein the presence of one, two or three of the following is indicative of the presence of hypersensitive platelets:
(a) a high level of CD36 in the sample;
(b) a high level of GPVI in the sample; and/or
(c) the subject is at least 50 years of age.

6. A method of identifying a subject having increased or high platelet reactivity or sensitivity to a platelet activating substance, the method comprising assessing platelet reactivity or sensitivity to a platelet activating substance in a sample comprising platelets from the subject by the method of any one of claims 1-3;
wherein the presence of one, two or three of the following is indicative of the presence of increased or high platelet reactivity or sensitivity to a platelet activating substance:
(a) a high level of CD36 in the sample;
(b) a high level of GPVI in the sample; and/or
(c) the subject is at least 50 years of age.

7. A method of identifying a subject with increased risk of a disease, disorder or condition associated with increased platelet reactivity, the method comprising assessing platelet reactivity or sensitivity to a platelet activating substance in a sample comprising platelets from the subject by the method of any one of claims 1-3;
wherein the presence of one, two or three of the following is indicative that the subject has increased risk of a disease, disorder or condition associated with increased platelet reactivity or sensitivity to a platelet activating substance:
(a) a high level of CD36 in the sample;
(b) a high level of GPVI in the sample; and/or
(c) the subject is at least 50 years of age.

8. A method of identifying a subject suitable for treatment with an anti-platelet therapy, comprising assessing platelet reactivity or sensitivity to a platelet activating substance in a sample comprising platelets from the subject by the method of any one of claims 1-3;
wherein the presence of one, two or three of the following is indicative that the subject may be suitable for treatment with an anti-platelet therapy:
(a) a high level of CD36 in the sample;
(b) a high level of GPVI in the sample; and/or
(ci) the subject is at least 50 years of age.

9. A method of identifying a subject at decreased risk of bleeding, e.g., in response to treatment with an anti-platelet therapy, the method comprising assessing platelet reactivity or sensitivity to a platelet activating substance in a sample comprising platelets from the subject by the method of any one of claims 1-3;
wherein the presence of one, two or three of the following is indicative that the subject is at decreased risk of bleeding:
(a) a high level of CD36 in the sample;
(b) a high level of GPVI in the sample; and/or
(c) the subject is at least 50 years of age.

10. The method of any one of claims 5 to 9, further comprising a step of administering an anti-platelet therapy to the subject.

11. A method of treating a disease, disorder or condition associated with increased platelet reactivity, the method comprising:
(i) assessing platelet reactivity or sensitivity to a platelet activating substance in a sample comprising platelets from the subject by the method of any one of claims 1-3; and
(ii) administering an anti-platelet therapy to a subject having one, two or three of the following:
(a) a high level of CD36;
(b) a high level of GPVI; and/or
(c) the subject is at least 50 years of age.

12. The method of any one of claims 5 to 11, wherein the method determines the presence of:
(a) a high level of CD36 in the sample; and
(b) a high level of GPVI in the sample;
wherein the subject is at least 50 years of age.

13. The method of any one of claims 5 to 11, wherein the method determines the presence of all three of:
(a) a high level of CD36 in the sample;
(b) a high level of GPVI in the sample; and
(ci) the subject is at least 50 years of age.

14. An anti-platelet therapy for use in a method of treating a subject in need thereof, the method comprising:
(iii) assessing platelet reactivity or sensitivity to a platelet activating substance in a sample comprising platelets from the subject by the method of any one of claims 1-3; and
(ii) administering the anti-platelet therapy to a subject having one, two or three of the following:
(a) a high level of CD36 in the sample;
(b) a high level of GPVI in the sample; and/or
(c) the subject is at least 50 years of age.

15. An anti-platelet therapy for use in a method of treating a subject in need thereof, wherein the subject has:
(a) a high level of CD36 in their platelets;
(b) a high level of GPVI in their platelets; and/or
(ci) an age of at least 50 years.

16. The anti-platelet therapy for use according to claim 14 or 15, wherein the method is a method of treating a disease, disorder or condition associated with increased platelet reactivity.

17. The anti-platelet therapy for use according to any one of claims 14 to 16, wherein the subject has all three of:
(a) a high level of CD36;
(b) a high level of GPVI; and
(c) an age of at least 50 years.

18. A method of identifying a subject having hyposensitive platelets, the method comprising assessing platelet reactivity or sensitivity to a platelet activating substance in a sample comprising platelets from the subject by the method of any one of claims 1-3;
wherein the presence of one, two or three of the following is indicative of the presence of hyposensitive platelets:
(a) a low level of CD36 in the sample;
(b) a low level of GPVI in the sample; and/or
(c) the subject is less than 50 years of age.

19. A method of identifying a subject not suitable for treatment with an anti-platelet therapy, the method comprising assessing platelet reactivity or sensitivity to a platelet activating substance in a sample comprising platelets from the subject by the method of any one of claims 1-3;
wherein the presence of one, two or three of the following is indicative that the subject may be unsuitable for treatment with anti-platelet therapy:
(a) a low level of CD36 in the sample;
(b) a low level of GPVI in the sample; and/or
(c) the subject is less than 50 years of age.

20. A method of identifying a subject at increased risk of bleeding, e.g., in response to treatment with an anti-platelet therapy, the method comprising assessing platelet reactivity or sensitivity to a platelet activating substance in a sample comprising platelets from the subject by the method of any one of claims 1-3;
wherein the presence of one, two or three of the following is indicative that the subject is at increased risk of bleeding:
(a) a low level of CD36 in the sample;
(b) a low level of GPVI in the sample; and/or
(c) the subject is less than 50 years of age.

21. The method of any one of claims 18 to 20, wherein the method determines the presence of all three of:
(a) a low level of CD36 in the sample;
(b) a low level of GPVI in the sample; and
(c) the subject is less than 50 years of age.

22. The method of any one of claims 18 to 20, wherein the method determines the presence of:
(a) a low level of CD36 in the sample; and
(b) a low level of GPVI in the sample; and
wherein the subject is less than 50 years of age.

23. The method of any one of claims 1 to 13, or the anti-platelet therapy for use according to any one of claims 14 to 17, wherein:
(i) the subject is at least 50 years of age; or
(ii) the subject is at least 55 years of age.

24. The method of any one of claims 1 to 4 or 18 to 22, wherein:
(i) the subject is less than 50 years of age; or
(ii) the subject is less than 45 years of age.

25. The method of any one of claims 5 to 13 or 23, or the anti-platelet therapy for use according to any one of claims 14 to 17 or 23, wherein a high level of CD36 is identified as:
(i) a level of CD36 that is in the top 35^{th} percentile compared to a reference population;
(ii) a level of CD36 that is at least 15% higher than the level of CD36 in a control or reference sample comprising platelets; or
(iii) a platelet or a population of platelets expressing at least 8000 copies of CD36 per platelet.

26. The method of any one of claims 18 to 22 or 24 wherein a low level of CD36 is identified as:
(i) a level of CD36 that is in the bottom 65^{th} percentile compared to a reference population;
(ii) a level of CD36 that is at least 15% lower than the level of CD36 in a control or reference sample comprising platelets; or
(iii) a platelet or a population of platelets expressing fewer than 8000 copies of CD36 per platelet.

27. The method of any one of claims 5 to 13, 23 or 25, or the anti-platelet therapy for use according to any one of claims 14 to 17, 23 or 25, wherein a high level of GPVI is defined as:
(i) a level of GPVI that is in the top 50^{th} percentile compared to a reference population;
(ii) a level of GPVI that is at least 15% higher than the level of GPVI in a control or reference sample comprising platelets; or
(iii) a platelet or a population of platelets expressing at least 3000 copies of GPVI per platelet.

28. The method of any one of claims 18 to 22, 24 or 25, wherein a low level of GPVI is defined as:
(i) a level of GPVI that is in the bottom 50^{th} percentile compared to a reference population;
(ii) a level of GPVI that is at least 15% lower than the level of GPVI in a control or reference sample comprising platelets; or
(iii) a platelet or a population of platelets expressing fewer than 3000 copies of GPVI per

29. The method of any one of claims 1 to 13 or 18 to 28, or the anti-platelet therapy for use according to any one of claims 14, 23, 25 or 27, wherein the platelet activating substance is:
(i) CRP;
(ii) CRP-XL;
(iii) ADP;
(iv) TRAP-6;
(v) epinephrine;
(vi) selected from the group consisting of CRP, CRP-XL, tissue factor, collagen, vWF, PAF, ristocetin, arachidonic acid, ADP, TRAP-6, epinephrine, U46619, and thrombin; or
(vii) any two or more different platelet activating substances as defined in (i) to (vi).

30. The method of any one of claims 1 to 13 or 18 to 29, or the anti-platelet therapy for use according to any one of claims 14 to 17, 23, 25, 27 or 29, wherein the sample:
(i) consists of platelets from the subject;
(ii) comprises or consists of a population of platelets from the subject;
(iii) is a cell-free sample;
(iv) is whole blood; or
(v) is plasma.

31. The method according to any one of claims 1 to 13 or 18 to 30, or the anti-platelet therapy for use according to any one of claims 14 to 17, 23, 25, 27 or 29 to 30, wherein the sample is from a subject that has not received anti-platelet therapy.

32. The method according to any one of claims 1 to 13 or 18 to 31, or the anti-platelet therapy for use according to any one of claims 14 to 17, 23, 25, 27 or 29 to 31, wherein the subject has no previous history of a disease, disorder or condition associated with increased platelet reactivity, optionally selected from: a cardiovascular disease, a metabolic or endocrine disease, an abnormal blood condition, an abnormal platelet count, a cancer, a response to a bacterial and/or a viral infection, and/or inflammation.

33. The method of claim 32, or the anti-platelet therapy for use according to claim 32, wherein the subject has no previous history of cardiovascular disease.

34. The method according to any one of claims 1 to 13 or 18 to 33, or the anti-platelet therapy for use according to any one of claims 14 to 17, 23, 25, 27 or 29 to 33, wherein the subject has, or is at risk of, a disease, disorder or condition selected from the following: a cardiovascular disease, a metabolic or endocrine disease, an abnormal blood condition, an abnormal platelet count, a cancer, a response to a bacterial and/or a viral infection, and/or inflammation.

35. The method according to any one of claims 1 to 13 or 18 to 34, or the anti-platelet therapy for use according to any one of claims 14 to 17, 23, 25, 27 or 29 to 34, wherein the subject is, or will be: receiving blood thinning medication, undergoing surgery, and/or a blood donor.

36. The method according to claim 7 or 11, or the anti-platelet therapy for use according to claim 16, wherein the disease, disorder or condition associated with increased platelet reactivity is selected from: a cardiovascular disease, a metabolic or endocrine disease, an abnormal blood condition, an abnormal platelet count, a cancer, a response to a bacterial and/or a viral infection, and/or inflammation.

37. The method according to any one of claims 32 to 34, or 36, or the anti-platelet therapy for use according to any one of claims 32 to 34, or 36, wherein the cardiovascular disease is selected from the group consisting of a thrombosis, a myocardial infarction, a stroke, a peripheral arterial disease, an aortic disease, and/or a coronary heart disease.

38. The method according to any one of claims 4, 8 to 11, 19, 31, or any claim dependent thereon, or the anti-platelet therapy for use according to any one of claims 14 to 17, 31, or any claim dependent thereon, wherein the anti-platelet therapy is:
(a) a platelet agglutination inhibitor;
(b) a platelet aggregation inhibitor; and/or
(c) selected from the group consisting of: aspirin, dipyridamole, cyclooxygenase inhibitors (for example, masoprocol, indomethacin, nabumetone, celecoxib, tolmetin, rofecoxib, piroxicam, fenoprofen, valdecoxib, and/or diclofenac), ADP receptor (P2Y) antagonists (for example, clopidogrel, prasugrel, ticagrelor, and/or cangrelor), GPVI inhibitors (for example, glenzocimab, revacept, honokiol, losartan, and/or tussilagone), and/or a drug that targets another platelet receptor, such as a GPIIbIIIa inhibitor (for example, tirofiban and/or eptifibatide), and/or a non-steroidal anti-inflammatory drug (NSAID).

39. The method according to any one of claims 1 to 13 or 18 to 38, or the anti-platelet therapy for use according to any one of claims 14 to 17, 23, 25, 27 or 29 to 38, wherein the level of CD36 and/or the level of GPVI is determined by:
(i) an ELISA;
(ii) a lateral flow test;
(iii) flow cytometry;
(iv) an immunological assay;
(v) an antibody assay; or
(vi) a two-site antibody assay
(vii) quantitative mass spectrometry.
